(19) United States
(12) Patent Application Publication
(10) Pub. No.: US 2007/0274926 A1
(43) Pub. Date: Nov. 29, 2007

(54) Method of inhibiting the transmission of viruses

(75) Inventors: Janice L. Fuls, Fountain Hills, AZ (US); James Dalton, Scottsdale, AZ (US); Priscilla S. Fox, Phoenix, AZ (US); Harry E. Towner, Scottsdale, AZ (US); Nancy D. Rodgers, Chandler, AZ (US); Daniel E. Pedersen, Cottage Grove, MN (US); John J. Rolando, Woodbury, MN (US); Richard K. Staub, Lakeville, MN (US)

Correspondence Address: MARSHALL, GERSTEIN & BORUN LLP (DIAL), 233 S. WACKER DRIVE, 6300 SEARS TOWER, CHICAGO, IL, 60606, US

(73) Assignee: THE DIAL CORPORATION, Scottsdale, AZ (US)

(21) Appl. No.: 11/805,391

(22) Filed: May 23, 2007

Related U.S. Application Data
(60) Provisional application No. 60/811,354, filed on Jun. 6, 2006.
(60) Provisional application No. 60/808,985, filed on May 26, 2006.

(51) Int. Cl. A61K 9/12; A61K 31/66; A61K 31/185; A61K 31/19; A61K 31/045

(52) U.S. Cl. 424/45; 514/553; 514/557; 514/724; 514/75

(57) ABSTRACT

A method of imparting a rapid and a persistent antiviral activity against viral-mediated diseases is disclosed. The method includes applying an antimicrobial compositions containing (a) a disinfecting alcohol, (b) an organic acid, and (c) water, and having a pH of about 5 or less, to at least one of the nose, nasal passages, and surrounding facial areas of skin of an individual.

### CROSS-REFERENCE TO RELATED APPLICATIONS

This application claims the benefit of U.S. Provisional Patent Application No. 60/808,985, filed May 26, 2006, and U.S. Provisional Patent Application No. 60/811,354, filed Jun. 6, 2006.

### FIELD OF THE INVENTION

The present invention relates to antimicrobial compositions having a rapid and persistent antiviral effectiveness, and application of the same to the nose area of the face to inhibit the transmission of viruses to the nasal mucosa and the respiratory tract. More particularly, the present invention relates to antimicrobial compositions comprising (a) a disinfecting alcohol and (b) an organic acid. The combination of (a) and (b) can provide a synergistic reduction in Gram negative and Gram positive bacteria and/or can synergistically inactivate or destroy viruses, such as rhinoviruses and rotaviruses, based on the log P (water-octanol partition coefficient) of the organic acid. The compositions have a pH of about 5 or less, and provide a substantial reduction in nonenveloped viral populations, and in Gram negative and Gram positive bacterial populations, within one minute after application of the composition to a nose and/or surrounding facial areas. In some embodiments, the compositions provide a barrier layer, or film, of the organic acid on a treated nose and facial area to impart a persistent antiviral activity to the nose and facial area.

### BACKGROUND OF THE INVENTION

Human health is impacted by a variety of microbes encountered on a daily basis. In particular, contact with various microbes in the environment can lead to an illness, possibly severe, in mammals. For example, microbial contamination can lead to a variety of illnesses, including, but not limited to, food poisoning, a streptococcal infection, anthrax (cutaneous), athlete's foot, cold sores, conjunctivitis (“pink eye”), coxsackievirus (hand-foot-mouth disease), croup, diphtheria (cutaneous), ebolic hemorrhagic fever, and impetigo.

It is known that washing body parts (e.g., hand washing) and hard surfaces (e.g., countertops and sinks) can significantly decrease the population of microorganisms, including pathogens. Therefore, cleaning skin and other animate and inanimate surfaces to reduce microbial populations is a first defense in removing such pathogens from these surfaces, and thereby minimizing the risk of infection.

Viruses are one category of pathogens of primary concern. Viral infections are among the greatest causes of human morbidity, with an estimated 60% or more of all episodes of human illness in developed countries resulting from a viral infection. In addition, viruses infect virtually every organism in nature, with high virus infection rates occurring among all mammals, including humans, pets, livestock, and zoo specimens.

Viruses exhibit an extensive diversity in structure and life cycle. A detailed description of virus families, their structures, life cycles, and modes of viral infection is discussed in Fundamental Virology, 4th Ed., Eds. Knipe & Howley, Lippincott Williams & Wilkins, Philadelphia, Pa., 2001.

Simply stated, virus particles are intrinsic obligate parasites, and have evolved to transfer genetic material between cells and encode sufficient information to ensure their propagation. In a most basic form, a virus consists of a small segment of nucleic acid encased in a simple protein shell. The broadest distinction between viruses is the enveloped and nonenveloped viruses, i.e., those that do or do not contain, respectively, a lipid-bilayer membrane.

Viruses propagate only within living cells. The principal obstacle encountered by a virus is gaining entry into the cell, which is protected by a cell membrane of thickness comparable to the size of the virus. In order to penetrate a cell, a virus first must become attached to the cell surface. Much of the specificity of a virus for a certain type of cell lies in its ability to attach to the surface of that specific cell. Durable contact is important for the virus to infect the host cell, and the ability of the virus and the cell surface to interact is a property of both the virus and the host cell. The fusion of viral and host-cell membranes allows the intact viral particle, or, in certain cases, only its infectious nucleic acid to enter the cell. Therefore, in order to control a viral infection, it is important to rapidly kill a virus that contacts the skin, and ideally to provide a persistent antiviral activity on the skin in order to control viral infections.

For example, rhinoviruses, influenza viruses, and adenoviruses are known to cause respiratory infections. Rhinoviruses are members of the picornavirus family, which is a family of “naked viruses” that lack an outer envelope. The human rhinoviruses are so termed because of their special adaptation to the nasopharyngeal region, and are the most important etiological agents of the common cold in adults and children. Officially there are 102 rhinoviruses serotypes. Most of the picornaviruses isolated from the human respiratory system are acid labile, and this lability has become a defining characteristic of rhinoviruses.

Rhinovirus infections are spread from person to person by direct contact with virus-contaminated respiratory secretions. It has been demonstrated that contaminated persons have a propensity to contaminate their hands and environmental objects by these respiratory secretions. Typically, this contamination is in the form of physical contact with a contaminated surface, rather than via inhalation of airborne viral particles.

Rhinovirus can survive on environmental surfaces for hours after initial contamination, and infection is readily transmitted by finger-to-finger contact, and by contaminated environmental surface-to-finger contact, if the newly contaminated finger then is used to rub an eye or touch the nasal area. Therefore, virus contamination of skin and environmental surfaces should be minimized to reduce the risk of transmitting the infection to the general population. Because a substantial proportion of rhinovirus colds are transmitted by direct contact from virus-contaminated hands or objects, it is possible to lower the risk of respiratory infection by inactivating virus on hands and surfaces, including the nose and nearby facial areas that the hands contact.

Several gastrointestinal infections also are caused by viruses, particularly rotaviruses and noroviruses. For example, Norwalk virus causes nausea, vomiting (sometimes accompanied by diarrhea), and stomach cramps. This infection typically is spread from person to person by direct contact. Acute hepatitis A viral infection similarly can be spread by direct contact between one infected person and a nonimmune individual by hand-to-hand, hand-to-mouth, or aerosol droplet transfer, or by indirect contact when an uninfected individual comes into contact with a hepatitis A virus-contaminated solid object.

Noroviruses are estimated to cause 23 million cases of acute gastroenteritis in the United States per year, and are the leading cause of gastroenteritis in the United States. Of viruses, only the common cold is reported more often than viral gastroenteritis (norovirus). Norovirus causes nausea, vomiting (sometimes accompanied by diarrhea), and stomach cramps. This infection typically is spread from person to person by direct contact.

Noroviruses are very highly contagious and can spread easily from person to person. Both stool (feces) and vomit are infectious. It is theorized that an inoculum of as few as 10 viral particles may be sufficient to infect an individual. Noroviruses are transmitted primarily through the fecal-oral route, either by consumption of fecally contaminated food or water, or by direct person-to-person spread. Environmental and fomite contamination also can act as a source of infection.

People can become infected with the norovirus in several ways, including, eating food or drinking liquids that are contaminated with norovirus; touching surfaces or objects contaminated with norovirus, and then placing their hands in their mouths; or having direct contact with another person who is infected and showing symptoms (for example, when caring for someone who is ill, or sharing foods or eating utensils with someone who is ill). During outbreaks of norovirus gastroenteritis, several modes of transmission have been documented, for example, initial foodborne transmission in a restaurant, followed by secondary person-to-person transmission to household contacts. No evidence suggests that norovirus infection occurs through the respiratory system.

A rotavirus also is a virus that is stable in the environment. Rotavirus infection is an infection of the digestive tract, and is the most common cause of severe diarrhea among children, resulting in over 50,000 hospitalizations yearly in the U.S. alone. Rotaviral infections are particularly problematic in close communities, such as child care facilities, geriatric facilities, family homes, and children's hospitals.

The most common mode of transmitting rotavirus is person to person spread through contaminated hands, but transmission also can occur through ingestion of contaminated water or food, or through contact with contaminated surfaces. The rotavirus then enters the body through contact with the mouth.

It is known that washing hands and hard surfaces with soap and/or other cleansers does not kill rotavirus, but helps prevent its spread. An oral rotavirus vaccine has been approved for use in children in the U.S., but its use is not recommended because of a severe adverse side effect. Because no other effective way to eliminate rotavirus, or its spread, is currently available, workers in close communities, especially those catering to children, must adhere to strict hygienic practices to help curtail the spread of rotavirus. An improved composition having enhanced antiviral efficacy, including persistent antiviral efficacy, in inactivating rotaviruses would further curtail the spread of rotavirus infections.

Numerous other viral infections are spread similarly. The risk of transmitting such viral infections can be reduced significantly by inactivating or removing viruses from the hands and other skin and mucosal surfaces that the hands contact.

Common household phenol/alcohol disinfectants are effective in disinfecting contaminated environmental surfaces, but lack persistent virucidal activity. Hand washing is highly effective in disinfecting contaminated fingers, but again suffers from a lack of persistent activity. These shortcomings illustrate the need for improved virucidal compositions having a persistent activity against viruses, such as rhinoviruses, rotaviruses, and noroviruses.

Antimicrobial personal care compositions are known in the art. In particular, antibacterial cleansing compositions, which typically are used to cleanse the skin and to destroy bacteria present on the skin, especially the hands, arms, and face of the user, are well-known commercial products.

Antibacterial compositions are used, for example, in the health care industry, food service industry, meat processing industry, and in the private sector by individual consumers. The widespread use of antibacterial compositions indicates the importance consumers place on controlling bacteria populations on skin. The paradigm for antibacterial compositions is to provide a substantial and broad spectrum reduction in bacterial populations quickly and without adverse side effects associated with toxicity and skin irritation. Such antibacterial compositions are disclosed in U.S. Pat. Nos. 6,107,261 and 6,136,771, each incorporated herein by reference.

One class of antibacterial personal care compositions is the hand sanitizer gels. This class of compositions is used primarily by medical personnel to disinfect the hands and fingers. A hand sanitizer gel is applied to, and rubbed into, the hands and fingers, and the composition is allowed to evaporate from the skin.

Hand sanitizer gels contain a high percentage of an alcohol, like ethanol. At the high percent of alcohol present in the gel, the alcohol itself acts as a disinfectant. In addition, the alcohol quickly evaporates to obviate wiping or rinsing skin treated with the sanitizer gel. Hand sanitizer gels containing a high percentage of an alcohol, i.e., about 40% or greater by weight of the composition, do not provide a persistent bacterial kill.

Antibacterial cleansing compositions typically contain an active antibacterial agent, a surfactant, and various other ingredients, for example, dyes, fragrances, pH adjusters, thickeners, skin conditioners, and the like, in an aqueous and/or alcoholic carrier. Several different classes of antibacterial agents have been used in antibacterial cleansing compositions. Examples of antibacterial agents include a bisguanidine (e.g., chlorhexidine digluconate), diphenyl compounds, benzyl alcohols, trihalocarbanilides, quaternary ammonium compounds, ethoxylated phenols, and phenolic compounds, such as halo-substituted phenolic compounds, like PCMX (i.e., p-chloro-m-xylenol) and triclosan (i.e., 2,4,4′-trichloro-2′-hydroxydiphenylether). Antimicrobial compositions based on such antibacterial agents exhibit a wide range of antibacterial activity, ranging from low to high, depending on the microorganism to be controlled and the particular antibacterial composition.

Most commercial antibacterial compositions generally offer a low to moderate antibacterial activity, and no reported antiviral activity. Antimicrobial activity is assessed against a broad spectrum of microorganisms, including both Gram positive and Gram negative microorganisms. The log reduction, or alternatively the percent reduction, in microbial populations provided by the antimicrobial composition correlates to antibacterial activity. A 1-3 log reduction is preferred, a log reduction of 3-5 is most preferred, whereas a log reduction of less than 1 is least preferred, for a particular contact time, generally ranging from 15 seconds to 5 minutes. Thus, a highly preferred antimicrobial composition exhibits a 3-5 log reduction against a broad spectrum of microorganisms in a short contact time.

Virus control poses a more difficult problem than bacterial control. By sufficiently reducing bacterial populations, the risk of bacterial infection is reduced to acceptable levels. Therefore, a rapid antibacterial kill is desired. With respect to viruses, however, not only is a rapid kill desired, but a persistent antiviral activity also is required. This difference is because merely reducing a virus population is insufficient to reduce infection. For example, in the case of a norovirus, about 10 virus particles can cause an infection. In theory, a single virus can cause infection. Therefore, an essentially total, and persistent, antiviral activity is required, or at least desired, for an effective antiviral cleansing composition.

U.S. Pat. No. 6,110,908 discloses a topical antiseptic containing a C₂₋₃ alcohol, a free fatty acid, and zinc pyrithione.

U.S. Pat. No. 5,776,430 discloses a topical antimicrobial cleaner containing chlorhexidine and an alcohol. The compositions contain about 50% to 60%, by weight, denatured alcohol and about 0.65% to 0.85%, by weight, chlorhexidine. The composition is applied to the skin, scrubbed into the skin, then rinsed from the skin.

European Patent Application 0 604 848 discloses a gel-type hand disinfectant containing an antimicrobial agent, 40% to 90% by weight of an alcohol, and a polymer and a thickening agent in a combined weight of not more than 3% by weight. The gel is rubbed into the hands and allowed to evaporate to provide disinfected hands. The disclosed compositions often do not provide immediate sanitization and do not provide persistent antimicrobial efficacy.

In general, hand sanitizer gels typically contain: (a) at least 60% by weight ethanol or a combination of lower alcohols, such as ethanol and isopropanol, (b) water, (c) a gelling polymer, such as a crosslinked polyacrylate material, and (d) other ingredients, such as skin conditioners, fragrances, and the like. Hand sanitizer gels are used by consumers to effectively sanitize the hands, without, or after, washing with soap and water, by rubbing the hand sanitizer gel on the surface of the hands. Current commercial hand sanitizer gels rely on high levels of alcohol for disinfection and evaporation, and thus suffer from disadvantages. Specifically, because of the volatility of ethanol, the primary antimicrobial agent does not remain on the skin after use, thus failing to provide a persistent antimicrobial effect.

At alcohol concentrations below 60%, ethanol is not recognized as an antiseptic. Thus, in compositions containing less than 60% alcohol, an additional antimicrobial compound is present to provide antimicrobial activity. Prior disclosures, however, have not addressed the issue of which composition ingredient in such an antimicrobial composition provides microbe control. Therefore, for formulations containing a reduced alcohol concentration, the selection of an antimicrobial agent that provides both a rapid antimicrobial effect and a persistent antimicrobial benefit is difficult.

U.S. Pat. Nos. 6,107,261 and 6,136,771 disclose highly effective antibacterial compositions containing a phenolic antimicrobial agent. These patents disclose compositions that solve the problem of controlling bacteria on skin and hard surfaces, but are silent with respect to controlling viruses.

U.S. Pat. Nos. 5,968,539; 6,106,851; and 6,113,933 disclose antibacterial compositions having a pH of about 3 to about 6. The compositions contain an antibacterial agent, an anionic surfactant, and a proton donor.

Antiviral compositions disclosed as inactivating or destroying pathogenic viruses, including rhinovirus, rotavirus, influenza virus, parainfluenza virus, respiratory syncytial virus, and Norwalk virus, also are known. For example, U.S. Pat. No. 4,767,788 discloses the use of glutaric acid to inactivate or destroy viruses, including rhinovirus. U.S. Pat. No. 4,975,217 discloses compositions containing an organic acid and an anionic surfactant, for formulation as a soap or lotion, to control viruses. U.S. Patent Publication 2002/0098159 discloses the use of a proton donating agent and a surfactant, including an antibacterial surfactant, to effect antiviral and antibacterial properties.

U.S. Pat. No. 6,034,133 discloses a virucidal hand lotion containing malic acid, citric acid, and a C₁₋₆ alcohol. U.S. Pat. No. 6,294,186 discloses combinations of a benzoic acid analog, such as salicyclic acid, and selected metal salts as being effective against viruses, including rhinovirus. U.S. Pat. No. 6,436,885 discloses a combination of known antibacterial agents with 2-pyrrolidone-5-carboxylic acid, at a pH of 2 to 5.5, to provide antibacterial and antiviral properties. U.S. Pat. No. 6,110,908 discloses a topical antiseptic containing a C₂₋₃ alcohol, a free fatty acid, and zinc pyrithione.

Organic acids in personal washing compositions also have been disclosed. For example, WO 97/46218 and WO 96/06152 disclose the use of organic acids or salts, hydrotropes, triclosan, and hydric solvents in a surfactant base for antimicrobial cleansing compositions. These publications are silent with respect to antiviral properties.

Hayden et al., Antimicrobial Agents and Chemotherapy, 26:928-929 (1984), discloses interrupting the hand-to-hand transmission of rhinovirus colds through the use of a hand lotion having residual virucidal activity. The hand lotions, containing 2% glutaric acid, were more effective than a placebo in inactivating certain types of rhinovirus. However, the publication discloses that the glutaric acid-containing lotions were not effective against a wide spectrum of rhinovirus serotypes.

A virucidal tissue designed for use by persons infected with the common cold, and including citric acid, malic acid, and sodium lauryl sulfate, is known. Hayden et al., Journal of Infectious Diseases, 152:493-497 (1985), however, reported that use of paper tissues, either treated with virus-killing substances or untreated, can interrupt the hand-to-hand transmission of viruses. Hence, no distinct advantage in preventing the spread of rhinovirus colds can be attributed to the compositions incorporated into the virucidal tissues.

An efficacious antimicrobial composition effective against both bacteria and viruses has been difficult to achieve because of the fundamental differences between a bacteria and a virus. Although a number of antimicrobial cleansing products currently exist, taking a variety of product forms (e.g., deodorant soaps, hard surface cleaners, and surgical disinfectants), such antimicrobial products typically incorporate antimicrobial agents, e.g., a phenolic compound, and/or harsh surfactants, which can dry out and irritate skin tissues. Ideally, personal cleansing products gently cleanse the skin, cause little or no irritation, and do not leave the skin overly dry after frequent use.

Accordingly, a need exists for an antimicrobial composition and method that is highly efficacious against a broad spectrum of microbes, including viruses and Gram positive and Gram negative bacteria, in a short time period, and wherein the composition and method can provide a persistent and broad spectrum antiviral activity, and is mild to the skin. A heightened level of persistent antiviral activity is provided by applying the present antimicrobial compositions to the nose and surrounding facial areas of an individual.

### SUMMARY OF THE INVENTION

The present invention is directed to antimicrobial compositions applied to the nose and surrounding facial areas that are continually touched by the fingers, including fingers contaminated with a virus. An application of a present composition provides a rapid and a persistent antiviral effectiveness, and a rapid and substantial reduction in Gram positive and Gram negative bacteria, in less than about one minute. More particularly, the present invention relates to antimicrobial compositions containing (a) a disinfecting alcohol, (b) an organic acid, and (c) water, wherein the composition has a pH of about 5 or less, and is applied to the nose and surrounding facial area. The composition can be applied topically to the skin on the nose or surrounding the nose, or internally to contact nasal mucosa.

In preferred embodiments, the composition is capable of providing a residual layer of the organic acid on treated skin or mucose. A present composition preferably is free of intentionally-added cleansing surfactants, such as anionic, cationic, and ampholytic surfactants.

Regardless of the log P of the organic acid, a present antimicrobial composition provides a rapid and persistent control of nonenveloped viruses, and a fast broad spectrum bacteria kill. The compostions also provide persistant control influenza viruses and noroviruses. In one embodiment, the organic acid has a water-octanol partition coefficient, expressed as log P, of less than one, and the composition exhibits a synergistic activity against nonenveloped viruses. In another embodiment, the organic acid has a log P of one or greater, and the composition exhibits a synergistic activity agent bacteria. In yet another embodiment, the organic acid comprises a first organic acid having a log P less than one and an organic acid having a log P of one or greater, and the composition exhibits a synergistic activity against both nonenveloped viruses and bacteria.

Accordingly, one aspect of the present invention is to provide an antimicrobial composition for application to the nose and surrounding facial areas, including nasal cavities, that is highly effective at killing a broad spectrum of bacteria, including Gram positive and Gram negative bacteria such as S. aureus, S. choleraesuis, E. coli, and K. pneumoniae, while simultaneously inactivating or destroying viruses harmful to human health, particularly nonenveloped viruses, like acid-labile viruses, and especially rhinoviruses, other acid-labile picornaviruses, and rotaviruses.

Another aspect of the present invention is to provide a liquid, antimicrobial composition for application to the nose and surrounding facial areas comprising:

(a) about 25% to 75%, by weight, of a disinfecting alcohol, like a C₁₋₆ alcohol;

(b) a virucidally effective amount of one or more organic acid; and

(c) water,

wherein the composition has a pH of about 5 or less.

In preferred embodiments, the composition provides an essentially continuous layer or film of the organic acid on treated skin or mucosa to impart a persistent antiviral activity to the treated skin or mucosa. In other preferred embodiments, the composition is free of an intentionally-added surfactant.

Yet another aspect of the present invention is to provide an antimicrobial composition for application to the nose and surrounding facial areas that exhibits a substantial, broad spectrum, and persistent virus control, and a substantial and broad spectrum bacteria control.

Another aspect of the present invention is to provide an antimicrobial composition having antibacterial and antiviral activity comprising (a) a disinfecting alcohol and (b) an organic acid that is substantive to the skin and the nasal mucosa and that fails to penetrate the skin and the nasal mucosa, for example, hydrophobic monocarboxylic acids, polycarboxylic acids, polymeric acids having a plurality of carboxylic, phosphate, sulfonate, and/or sulfate moieties, or mixtures thereof, and (c) water, wherein the composition has a pH of about 5 or less. Such organic acids typically have a log P of less than one, and the compositions are effective against a broad spectrum of bacteria and exhibit a synergistic activity against nonenveloped viruses.

Still another aspect of the present invention is to provide an antimicrobial composition for application to the nose and surrounding facial areas, and having antibacterial and antiviral activity, comprising (a) a disinfecting alcohol and (b) an organic acid selected from the group consisting of monocarboxylic acids, polycarboxylic acids, polymeric acids having a plurality of carboxylic, phosphate, sulfonate, and/or sulfate moieties, or mixtures thereof, and (c) water, wherein the composition has a pH of about 5 or less, and the organic acid has a log P of one or greater. These compositions provide an effective and persistent control of nonenveloped viruses and exhibit a synergistic activity against Gram positive and Gram negative bacteria.

Another aspect of the present invention is to provide an antimicrobial composition comprising an organic acid that is substantive to the skin, and/or that fails to penetrate the skin, and/or that resists rinsing from the skin, and/or that forms an essentially continuous barrier layer on the skin. Such organic acids typically have a log P of less than one, and the compositions are effective against a broad spectrum of bacteria and exhibit a synergistic activity against viruses. The persistent antiviral activity is attributed, in part, to a residual layer or film of the organic acid on treated skin or mucosa, which resists removal from the skin after several rinsings, and during normal daily routines for a period of several hours.

Preferred compositions comprise one or more polycarboxylic acid, a polymeric acid, and a gelling agent. These compositions provide, an effective and persistent control of viruses and exhibit a synergistic activity against Gram positive and Gram negative bacteria.

Another aspect of the present invention is to provide an antimicrobial composition that exhibits a substantial, and preferably persistent, control of viruses, and has a pH of about 2 to about 5.

Yet another aspect of the present invention is to provide an antimicrobial composition that exhibits a log reduction against Gram positive bacteria (i.e., S. aureus) of at least 2 after 30 seconds of contact.

Still another aspect of the present invention is to provide an antimicrobial composition that exhibits a log reduction against Gram negative bacteria (i.e., E. coli) of at least 2.5 after 30 seconds of contact.

Another aspect of the present invention is to provide an antimicrobial composition that resists rinsing from the skin, e.g., at least 50%, at least 60%, and preferably at least 70% of the nonvolatile components of an applied composition remains on skin or mucosa after three water rinsings and an effective antiviral amount of the composition remains on the skin after ten water rinsings.

Another aspect of the present invention is to provide an antimicrobial composition for application to the nose and surrounding areas, that exhibits a log reduction against nonenveloped viruses, such as acid-labile viruses, including rhinovirus serotypes, such as Rhinovirus 1a, Rhinovirus 2, Rhinovirus 14, and Rhinovirus 4, and rotavirus serotypes, such as Rotavirus Wa, of at least 4 after 30 seconds of contact. The antimicrobial composition also provides a log reduction against nonenveloped viruses of about 3 for at least about five hours, and at least 2 for about six hours, after application with a 30 second contact time. In some embodiments, the antimicrobial composition provides a log reduction of 2 against nonenveloped viruses for up to about eight to twelve hours.

Another aspect of the present invention is to provide consumer products based on an antimicrobial composition for application to the nose and surrounding facial area, including nasal passages, for example, sprays, drops, ointments, washings, pastes, gels, sticks, swabs, wipes, nasal packings, and the like. Preferably, the composition forms a coating on the nose and surrounding facial area to provide a persistent antiviral activity. The compositions are esthetically pleasing and nonirritating to the nose and surrounding facial area, including the nasal mucosa, and provide an essentially continuous residual film or layer of the nonvolatile composition components, e.g., the organic acid.

A further aspect of the present invention is to provide a method of quickly controlling a wide spectrum of viruses, and Gram positive and Gram negative bacteria populations, on nasal mucosa, including human nasal mucosa, by contacting nasal cavities with a composition of the present invention, and allowing the composition to remain in contact with the nasal mucosa to provide a persistent control of viruses.

Still another aspect of the present invention is to provide a method treating or preventing virus-mediated diseases and conditions caused by rhinoviruses, picornaviruses, adenoviruses, herpes viruses, respiratory syncytial viruses (RSV), coronaviruses, enteroviruses, and other nonenveloped viruses, by applying a composition of the present invention to skin on and surrounding the nose.

Yet another aspect of the present invention is to provide a composition and method of interrupting transmission of a virus from animate surfaces, like human fingers, to human nasal mucosa. Especially provided is a method and composition for controlling the transmission of nonenveloped viruses, particularly rhinovirus, either by preventing viruses from entering nasal cavities or by effectively controlling viruses introduced into nasal cavities, for example, by contact with a contaminated hand or finger, and continuing to control the viruses for a period of about four or more hours, and up to about 12 hours, after application of the composition to the skin on or surrounding the nose, including a nasal cavity.

Another aspect of the present invention is to provide a dispensing device, preferably hand held, comprising a reservoir of a present antimicrobial composition, wherein the device is adapted to dispense the composition onto the nose and surrounding facial area, and/or into a human nostril.

Still another aspect of the present method is to provide a method for the treatment or prophylaxis of the common cold, in a subject in need thereof, comprising administering a present composition to the skin on or surrounding the nose, including a nasal cavity.

These and other novel aspects and advantages of the present invention are set forth in the following, nonlimiting detailed description of the preferred embodiments.

### BRIEF DESCRIPTION OF THE FIGURES

FIGS. 1a and 1b are reflectance micrographs showing a barrier layer of nonvolatile components on a surface provided by application of a composition of the present invention to the surface, and

FIGS. 1c and 1d are reflectance micrographs showing the absence of a barrier layer on a surface after application of a control composition to the surface.

### DETAILED DESCRIPTION OF THE PREFERRED EMBODIMENTS

Personal care products incorporating an active antimicrobial agent have been known for many years. Since the introduction of antimicrobial personal care products, many claims have been made that such products provide antimicrobial properties. To be most effective, an antimicrobial composition should provide a high log reduction against a broad spectrum of organisms in as short a contact time as possible. Ideally, the composition also should inactivate viruses.

As presently formulated, most commercial liquid antibacterial soap compositions provide a poor to marginal time kill efficacy, i.e., rate of killing bacteria. These compositions do not effectively control viruses.

Antimicrobial hand sanitizer compositions typically do not contain a surfactant and rely upon a high concentration of an alcohol to control bacteria. The alcohols evaporate and, therefore, cannot provide a persistent bacterial control. The alcohols also can dry and irritate the skin.

Most current products especially lack efficacy against Gram negative bacteria, such as E. coli, which are of particular concern to human health. Compositions do exist, however, that have an exceptionally high broad spectrum antibacterial efficacy, as measured by a rapid kill of bacteria (i.e., time kill), which is to be distinguished from persistent kill. These products also lack a sufficient antiviral activity.

The present antimicrobial compositions provide excellent broad spectrum antiviral and antibacterial efficacy and significantly improve antiviral efficacy compared to prior compositions that incorporate a high percentage of an alcohol, i.e., 40% or greater, by weight. The basis of this improved efficacy is (a) the discovery that a combination of a disinfecting alcohol and an organic acid, and especially an organic acid having a log P of less than about 1, substantially improves antiviral efficacy, and (b) the pH of a surface after application of the composition to the surface.

An important aspect of the present invention is to maintain a low skin pH for an extended time to provide a persistent antiviral activity. In preferred embodiments, this is achieved by forming an essentially continuous film of the nonvolatile composition components on the skin, which provides a reservoir of the organic acids to maintain a low skin pH.

The term “essentially continuous film” means that a residue of the nonvolatile components of the composition in the form of a barrier layer is present on at least 50%, at least 60%, at least 70%, or at least 80%, preferably at least 85% or at least 90%, and more preferably at least 95%, of the area of the treated surface area. An “essentially continuous” film is demonstrated in the reflectance micrographs of the figures, which are discussed hereafter. The term “essentially continuous film” as used herein is synonymous with the term “essentially continuous layer”, “barrier layer”, and “barrier film”.

A disinfecting alcohol and an organic acid having a log P of less than one act synergistically to control nonenveloped viruses. A disinfecting alcohol and an organic acid having a log P of one or greater act synergistically to substantially improve antibacterial efficacy. A combination of a first organic acid having a log P less than one and a second organic acid having a log P of one or greater, with a disinfecting alcohol, provides a synergistic improvement in the control of nonenveloped viruses and Gram positive and Gram negative bacteria.

Although compositions containing an antimicrobial agent, like triclosan, have demonstrated a rapid and effective antibacterial activity against Gram positive and Gram negative bacteria, control of viruses has been inadequate. Virus control on skin and inanimate surfaces is very important in controlling the transmission of numerous diseases.

For example, rhinoviruses are the most significant microorganisms associated with the acute respiratory illness referred to as the “common cold.” Other viruses, such as parainfluenza viruses, respiratory syncytial viruses (RSV), enteroviruses, and coronaviruses, also are known to cause symptoms of the “common cold,” but rhinoviruses are theorized to cause the greatest number of common colds. Rhinoviruses also are among the most difficult of the cold-causing viruses to control, and have an ability to survive on a hard dry surface for more than four days. In addition, most viruses are inactivated upon exposure to a 70% ethanol solution. However, rhinoviruses remain viable upon exposure to ethanol.

Because rhinoviruses are the major known cause of the common cold, it is important that a composition having antiviral activity is active against the rhinovirus. Although the molecular biology of rhinoviruses is now understood, finding effective methods for preventing colds caused by rhinoviruses, and for preventing the spread of the virus to noninfected subjects, has been fruitless.

It is known that iodine is an effective antiviral agent, and provides a persistent antirhinoviral activity on skin. In experimentally induced and natural cold transmission studies, subjects who used iodine products had significantly fewer colds than placebo users. This indicates that iodine is effective for prolonged periods at blocking the transmission of rhinoviral infections. Thus, the development of products that deliver both immediate and persistent antiviral activity would be effective in reducing the incidence of colds. Likewise, a topically applied composition that exhibits antiviral activity would be effective in preventing and/or treating diseases caused by other nonenveloped viruses, including acid-labile viruses, and by influenza viruses.

Virucidal means capable of inactivating or destroying a virus. As used herein, the term “persistent antiviral efficacy” or “persistent antiviral activity” means leaving a barrier residue or film of antiviral agents, including organic acids, on animate (e.g., skin) surfaces that provides significant antiviral activity for an extended time after application. The barrier residue or film can be continuous or essentially continuous, and resists removal from a treated surface during water rinsing.

A composition of the present invention provides a persistent antiviral efficacy, i.e., preferably a log reduction of at least 3, and more preferably a log reduction of at least log 4, against nonenveloped viruses, including acid-labile viruses, such as rhinovirus and rotavirus serotypes, within 30 seconds of contact with the composition. Antiviral activity is maintained for at least about 0.5 hour, preferably at least about one hour, and more preferably for at least about two hours, at least about three hours, or at least about four hours after contact with the composition. In some preferred embodiments, antiviral activity is maintained for about six to about twelve hours after contact with the composition. The persistent antiviral activity is attributed, at least in part, to the reservoir of organic acids present in the barrier layer or film of the composition on a treated surface. The methodology utilized to determine a persistent antiviral efficacy is discussed below.

The term “nose and surrounding area” means the external portions of the nose, including the infratip lobule, columella, alar sidewalls, facets (or soft tissue triangles), the nostril sill, the colomella-labiel angle or junction, the alar-facial groove or junction, the tip defining points, and other facial skin surrounding the nose, such as the skin between the upper lip extending to and above the nasal opening, and the cheek area near the nose. The term also includes the internal surfaces of the nose, including the nasal cavities up to the sinuses.

The antimicrobial compositions utilized in the present invention are highly effective in providing a rapid and broad spectrum control of bacteria, and a rapid and persistent control of nonenveloped viruses. The highly effective compositions comprise a disinfecting alcohol and a virucidally effective amount of an organic acid. Preferred embodiments comprise at least one of a polymeric acid and a gelling agent. Other preferred embodiments comprise a polymeric acid and a gelling agent.

The disinfecting alcohol and an organic acid having a log P of less than about 1 act synergistically to control a broad spectrum of nonenveloped viruses. The disinfecting alcohol and an organic acid having a log P of 1 or greater act synergistically to control a broad spectrum of bacteria. A composition containing a first organic acid having a log P of less than one and a second organic acid having a log P of one or greater act synergistically to control a broad spectrum of nonenveloped viruses and a broad spectrum of Gram positive and Gram negative bacteria.

The compositions are surprisingly mild to the skin and external and internal portions of the nose, including the nasal passages. Thus, mild and effective compositions that solve the problem of bacterial and viral control are provided to consumers.

The present compositions provide an effective and persistent inactivation of nonenveloped viruses. Nonenveloped viruses include, but are not limited to, adenoviruses, papovaviruses, parvoviruses, astroviruses, caliciviruses (including Norwalk virus), and picornaviruses (including rhinovirus, polio virus, and hepatitis A virus). The compositions also are effective against influenza viruses, noroviruses, and rotoviruses (including rotovirus gastroenteritis).

The antimicrobial compositions are highly efficacious in sprays, drops, ointments, rinses, pastes, gels, sticks, and packings. The antimicrobial compositions also can be applied to wipes and swabs for topical application. The antimicrobial compositions, after application to the nose and surrounding facial areas, including nasal passages, efficaciously and rapidly disinfect skin and nasal mucosa that is infected or contaminated with Gram negative bacteria, Gram positive bacteria, and nonenveloped viruses (e.g., rhinoviruses and rotaviruses). The compositions also provide a persistent antiviral effectiveness against future viral contamination resulting from contact with an infected surface, such as a finger.

As illustrated in the following nonlimiting embodiments, an antimicrobial composition comprises: (a) about 25% to about 75%, by weight, of a disinfecting alcohol; (b) a virucidally effective amount of an organic acid; and (c) water. The compositions have a pH of less than about 5. In preferred embodiments, the composition contains an optional gelling agent.

The compositions have a pH of less than about 5, and are capable of forming an essentially continuous film or layer of nonvolatile composition ingredients on a treated surface. The film or layer resists removal from the treated surface for several hours after application. In particular, an effective amount of composition ingredients remain on a treated surface after ten rinsings, and at least 50%, preferably at least 60%, and more preferably at least 70%, of the nonvolatile composition ingredients remains on a treated surface after three rinsings. As used herein, “rinsing” means gently rubbing treated skin under a moderate flow of tap water having a temperature of about 30° C. to about 40° C. for about 30 seconds, then air drying the skin.

The compositions exhibit a log reduction against Gram positive bacteria of about 2 after 30 seconds contact. The compositions also exhibit a log reduction against Gram negative bacteria of about 2.5 after 30 seconds contact. The compositions further exhibit a log reduction against nonenveloped viruses, including acid-labile viruses, such as rhinovirus and rotavirus serotypes of about 5 after 30 seconds contact, and a log reduction against these acid-labile viruses of at least 3 about five hours after contact, and at least about 2 about six to about twelve hours after contact. The compositions also are mild, and it is not necessary to rinse or wipe the compositions from treated areas. It is preferred that the composition is allowed to remain on the treated areas to provide an enhanced persistent antiviral activities.

In accordance with the invention, the antimicrobial composition can further comprise additional optional ingredients disclosed hereafter, like hydrotropes, polyhydric solvents, gelling agents, pH adjusters, vitamins, dyes, conditioners, and perfumes. The compositions preferably are free of intentionally-added cleansing surfactants, like anionic surfactants, i.e., contain 0% to about 0.5%, by weight, of compounds that exhibit surface activity.

The following ingredients are present in an antimicrobial composition used in a method of the present invention.

A. Disinfecting Alcohol

The antimicrobial compositions contain about 25% to about 75%, by weight, of a disinfecting alcohol. Preferred embodiments contain about 30% to about 75%, by weight, of a disinfecting alcohol. Most preferred embodiments contain about 30% to about 70%, by weight, of a disinfecting alcohol.

As used herein, the term “disinfecting alcohol” is a water-soluble alcohol containing one to six carbon atoms. Disinfecting alcohols include, but are not limited to, methanol, ethanol, propanol, and isopropyl alcohol.

B. Organic Acid

A present antimicrobial composition also contains an organic acid in a sufficient amount to control and inactivate viruses and bacteria on a surface contacted by the antimicrobial composition. The organic acid acts synergistically with the disinfecting alcohol to provide a rapid control of nonenveloped viruses and/or bacteria, and provides a persistent viral control.

In particular, an organic acid is present in the composition in a sufficient amount such that the pH of the animate surface, e.g., a nose or nostril contacted by the composition, is lowered to degree wherein a persistent viral control is achieved. This persistent viral control is enhanced when the composition is allowed to remain on the contacted nose or nasal mucosa. The organic acid remains at least partially undissociated in the composition, and remains so when the composition is diluted, or during application and rinsing.

Upon application to the nose, the pH of skin is sufficiently lowered such that a persistent viral control is achieved. In preferred embodiments, a residual amount of the organic acid remains on the treated areas, even after a rinsing step, in order to impart a persistent viral control. However, even if the organic acid is essentially completely rinsed or flushed from the treated areas, the surface pH has been sufficiently lowered to impart a viral control for at least 0.5 hour.

A preferred composition is a leave-on composition, i.e., is not intended to be rinsed from the skin. However, after three rinsings, at least 50% of nonvolatile composition ingredients remain on the skin or nasal mucosa, and an effective amount of the composition remains on the treated skin or nasal mucosa after ten rinsings.

Typically, an organic acid is included in a present composition in an amount of about 0.05% to about 15%, and preferably about 0.1% to about 10%, by weight of the composition. To achieve the full advantage of the present invention, the organic acid is present in an amount of about 0.15% to about 6%, by weight of the composition. In preferred embodiments, a mixture of organic acid is included in the composition. The amount of organic acid is related to the class of organic acid used, and to the identity of the specific acid or acids used.

An organic acid included in an antimicrobial composition preferably does not penetrate the surface to which it is applied, e.g., remains on the internal or external surface of the nose as opposed to penetrating the surface, and forms a layer of film on the skin or nasal mucosa, together with other nonvolatile composition ingredients, e.g., an optional gelling agent. The organic acid, therefore, preferably is a hydrophobic organic acid.

In one embodiment of the present invention, the organic acid has a log P of less than one, and preferably less than 0.75. To achieve the full advantage of the present invention, the organic acid has a log P of less than 0.5. In this embodiment, the disinfecting alcohol and organic acid act synergistically to provide an effective and persistent viral control.

In another embodiment, the organic acid has a log P of 1 or greater, for example, 1 to about 100. In this embodiment, the disinfecting alcohol and organic acid effectively control nonenveloped viruses and also act synergistically to control a broad spectrum of bacteria.

It is envisioned that, by incorporating a first organic acid having a log P of less than one and a second organic acid having a log P of 1 or greater into an antimicrobial composition, the first and second organic acids act synergistically with the disinfecting alcohol to provide a persistent control of nonenveloped viruses and a broad spectrum bacteria control.

As used herein, the term “log P” is defined as the log of the water-octanol partition coefficient, i.e., the log of the ratio P_{w}/Pₒ, wherein P_{w} is the concentration of an organic acid in water and Pₒ is the concentration of the organic acid in octanol, at equilibrium and 25° C. The water-octanol coefficient can be determined by the U.S. Environmental Protection Agency Procedure, “OPPTS 830.7560 Partition Coefficient (n-Octanol/Water), Generator Column Method” (1996).

Organic acids having a log P less than one typically are water insoluble, e.g., have a water solubility of less than about 0.5 wt % at 25° C. Organic acids having a log P of one or greater typically are considered water soluble, e.g., have a water solubility of at least 0.5 wt %, at 25° C.

The organic acid can comprise a monocarboxylic acid, a polycarboxylic acid, a polymeric acid having a plurality of carboxylic, phosphate, sulfonate, and/or sulfate moieties, or mixtures thereof. In addition to acid moieties, the organic acid also can contain other moieties, for example, hydroxy groups and/or amino groups. In addition, an organic acid anhydride can be used in an antimicrobial composition as the organic acid. Preferred organic acids are polycarboxylic acids, polymeric carboxylic acids, and mixtures thereof.

In one embodiment, the organic acid comprises a monocarboxylic acid having a structure RCO₂H, wherein R is C₁₋₆alkyl, hydroxyC₁₋₆alkyl, haloC₁₋₆alkyl, phenyl, or substituted phenyl. The alkyl groups can be substituted with phenyl groups and/or phenoxy groups, and these phenyl and phenoxy groups can be substituted or unsubstituted.

Nonlimiting examples of monocarboxylic acids useful in the antimicrobial compositions are acetic acid, propionic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof. Additional substituted benzoic acids are disclosed in U.S. Pat. No. 6,294,186, incorporated herein by reference. Examples of substituted benzoic acids include, but are not limited to, salicyclic acid, 2-nitrobenzoic acid, thiosalicylic acid, 2,6-dihydroxybenzoic acid, 5-nitrosalicyclic acid, 5-bromosalicyclic acid, 5-iodosalicyclic acid, 5-fluorosalicylic acid, 3-chlorosalicylic acid, 4-chlorosalicyclic acid, and 5-chlorosalicyclic acid.

In another embodiment, the organic acid comprises a polycarboxylic acid. The polycarboxylic acid contains at least two, and up to four, carboxylic acid groups. The polycarboxylic acid also can contain hydroxy or amino groups, in addition to substituted and unsubstituted phenyl groups. Preferably, the polycarboxylic acid has a water solubility of at least about 0.05%, by weight, at 25° C.

Nonlimiting examples of polycarboxylic acids useful in the present invention include malonic acid, succinic acid, glutaric acid, adipic acid, terephthalic acid, phthalic acid, pimelic acid, suberic acid, azelaic acid, sebacic acid, fumaric acid, maleic acid, tartaric acid, malic acid, citric acid, maleic acid, aconitic acid, and mixtures thereof.

Anhydrides of polycarboxylic and monocarboxylic acids also are organic acids useful in the antimicrobial compositions. Preferred anhydrides are anhydrides of polycarboxylic acids, e.g., phthalic anhydride. At least a portion of the anhydride is hydrolyzed to a carboxylic acid because of the pH of the composition. It is envisioned that an anhydride can be slowly hydrolyzed on a surface contacted by the composition, and thereby assist in providing a persistent antiviral activity.

In a third embodiment, the organic acid comprises a polymeric carboxylic acid, a polymeric sulfonic acid, a sulfated polymer, a polymeric phosphoric acid, or mixtures thereof. The polymeric acid has a molecular weight of about 500 g/mol to 10,000,000 g/mol, and includes homopolymers, copolymers, and mixtures thereof. The polymeric acid preferably is capable of forming a substantive film on a surface and has a glass transition temperature, T_{g}, of less than 25° C., preferably less than 20° C., and more preferably less than about 15° C. The glass transition temperature is the temperature at which an amorphous material, such as a polymer, changes from a brittle, vitreous state to a plastic state. The T_{g} of a polymer is readily determined by persons skilled in the art using standard techniques.

The polymeric acids are uncrosslinked or only very minimally crosslinked. The polymeric acids typically are prepared from ethylenically unsaturated monomers having at least one hydrophilic moiety, such as carboxyl, carboxylic acid anhydride, sulfonic acid, and sulfate. The polymeric acid can contain a comonomer, such as styrene or an alkene, to increase the hydrophobicity of the polymeric acid.

Examples of monomers used to prepare the polymeric organic acid include, but are not limited to:

(a) Carboxyl group-containing monomers, e.g., monoethylenically unsaturated mono- or polycarboxylic acids, such as acrylic acid, methacrylic acid, maleic acid, fumaric acid, crotonic acid, sorbic acid, itaconic acid, ethacrylic acid, α-chloroacrylic acid, α-cyanoacrylic acid, β-methylacrylic acid (crotonic acid), α-phenylacrylic acid, β-acryloxypropionic acid, sorbic acid, α-chlorosorbic acid, angelic acid, cinnamic acid, p-chlorocinnamic acid, β-stearylacrylic acid, citraconic acid, mesaconic acid, glutaconic acid, aconitic acid, tricarboxyethylene, and cinnamic acid;

(b) Carboxylic acid anhydride group-containing monomers, e.g., monoethylenically unsaturated polycarboxylic acid anhydrides, such as maleic anhydride; and

(c) Sulfonic acid group-containing monomers, e.g., aliphatic or aromatic vinyl sulfonic acids, such as vinylsulfonic acid, allylsulfonic acid, vinyltoluenesulfonic acid, styrenesulfonic acid, sulfoethyl(meth)acrylate, 2-acrylamido-2-methylpropane sulfonic acid, sulfopropyl(meth)acrylate, and 2-hydroxy-3-(meth)acryloxy propyl sulfonic acid.

The polymeric acid can contain other copolymerizable units, i.e., other monoethylenically unsaturated comonomers, well known in the art, as long as the polymer is substantially, i.e., at least 10%, and preferably at least 25%, acid group containing monomer units. To achieve the full advantage of the present invention, the polymeric acid contains at least 50%, and more preferably, at least 75%, and up to 100%, acid group containing monomer units. The other copolymerizable units, for example, can be styrene, an alkene, an alkyl acrylate, or an alkyl methacrylate. The polymeric acid also can be partially neutralized, which assists dispersion of the polymeric acid into a composition. However, a sufficient number of the acid groups remain unneutralized to reduce skin pH and impart a persistent antiviral activity.

A polymeric acid assists in forming a film or layer of residual organic acid on the skin, and assists further in forming a more continuous layer of residual organic acid on the skin or nasal mucosa. A polymeric acid typically is used in conjunction with a monocarboxylic acid and/or a polycarboxylic acid.

One preferred polymeric acid is a polyacrylic acid, either a homopolymer or a copolymer, for example, a copolymer of acrylic acid and an alkyl acrylate and/or alkyl methacrylate. Another preferred polymeric acid is a homopolymer or a copolymer of methacrylic acid.

Exemplary polymeric acids useful in the present invention include, but are not limited to:

| | (CARBOPOL 910, |
|---|---|
| | 934, 934P, 940, |
| | 941, ETD 2050; |
| | ULTREZ 10, 21) |
| | (CARBOPOL ETD |
| Carbomers | 2050) |
| Acrylates/C20-30 Alkyl Acrylate Crosspolymer | (ULTREZ 20) |
| Acrylates/Beheneth 25 Methacrylate Copolymer | (ACULYN 28) |
| Acrylates/Steareth 20 Methacrylate Copolymer | (ACULYN 22) |
| Acrylates/Steareth 20 Methacrylate Crosspolymer | (ACULYN 88) |
| Acrylates Copolymer | (CAPIGEL 98) |
| Acrylates Copolymer | (AVALURE AC) |
| Acrylates/Palmeth 25 Acrylate Copolymer | (SYNTHALEN |
| | 2000) |
| Ammonium Acrylate Copolymers | |
| Sodium Acrylate/Vinyl Alcohol Copolymer | |
| Sodium Polymethacrylate | |
| Acrylamidopropyltrimonium Chloride/Acrylates | |
| Copolymer | |
| Acrylates/Acrylamide Copolymer | |
| Acrylates/Ammonium Methacrylate Copolymer | |
| Acrylates/C10-30 Alkyl Acrylate Crosspolymer | |
| Acrylates/Diacetoneacrylamide Copolymer | |
| Acrylates/Octylacrylamide Copolymer | |
| Acrylates/VA Copolymer | |
| Acrylic Acid/Acrylonitrogens Copolymer | |

In a preferred embodiment, the organic acid comprises one or more polycarboxylic acid, e.g., citric acid, malic acid, tartaric acid, or a mixture of any two or all three of these acids, and a polymeric acid containing a plurality of carboxyl groups, for example, homopolymers and copolymers of acrylic acid or methacrylic acid.

C. Carrier

The carrier of the antimicrobial composition comprises water.

D. Optional Ingredients

An antimicrobial composition also can contain optional ingredients well known to persons skilled in the art. The particular optional ingredients and amounts that can be present in the composition are discussed hereafter.

The optional ingredients are present in a sufficient amount to perform their intended function and not adversely affect the antimicrobial efficacy of the composition, and in particular not adversely affect the synergistic effect provided by the disinfecting alcohol and organic acid, or a layer or film formed on treated skin or mucosa by the nonvolatile components of the composition. Optional ingredients typically are present, individually or collectively, from 0% to about 50%, by weight of the composition.

Classes of optional ingredients include, but are not limited to, hydrotropes, polyhydric solvents, gelling agents, dyes, fragrances, pH adjusters, thickeners, viscosity modifiers, chelating agents, skin conditioners, emollients, preservatives, buffering agents, antioxidants, chelating agents, opacifiers, and similar classes of optional ingredients known to persons skilled in the art.

A hydrotrope, if present at all, is present in an amount of about 0.1% to about 30%, and preferably about 1% to about 20%, by weight of the composition. To achieve the full advantage of the present invention, a composition can contain about 2% to about 15%, by weight, of a hydrotrope.

A hydrotrope is a compound that has an ability to enhance the water solubility of other compounds. A hydrotrope utilized in the present invention lacks surfactant properties, and typically is a short-chain alkyl aryl sulfonate. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

A polyhydric solvent, if present at all, is present in an amount of about 0.1% to about 30%, and preferably about 5% to about 30%, by weight of the composition. To achieve the full advantage of the present invention, the polyhydric solvent is present in an amount of about 10% to about 30% by weight of the composition. In contrast to a disinfecting alcohol, a polyhydric solvent contributes minimally, if at all, to the antimicrobial efficacy of the present composition.

The term “polyhydric solvent” as used herein is a water-soluble organic compound containing two to six, and typically two or three, hydroxyl groups. The term “water-soluble” means that the polyhydric solvent has a water solubility of at least 0.1 g of polyhydric solvent per 100 g of water at 25° C. There is no upper limit to the water solubility of the polyhydric solvent, e.g., the polyhydric solvent and water can be soluble in all proportions.

The term polyhydric solvent, therefore, encompasses water-soluble diols, triols, and polyols. Specific examples of hydric solvents include, but are not limited to, ethylene glycol, propylene glycol, glycerol, diethylene glycol, dipropylene glycol, tripropylene glycol, hexylene glycol, butylene glycol, 1,2,6-hexanetriol, sorbitol, PEG-4, and similar polyhydroxy compounds.

Other specific classes of optional ingredients include inorganic phosphates, sulfates, and carbonates as buffering agents; EDTA and phosphates as chelating agents; and acids and bases as pH adjusters.

Examples of preferred classes of optional basic pH adjusters are ammonia; mono-, di-, and tri-alkyl amines; mono-, di-, and tri-alkanolamines; alkali metal and alkaline earth metal hydroxides; and mixtures thereof. However, the identity of the basic pH adjuster is not limited, and any basic pH adjuster known in the art can be used. Specific, nonlimiting examples of basic pH adjusters are ammonia; sodium, potassium, and lithium hydroxide; monoethanolamine; triethylamine; isopropanolamine; diethanolamine; and triethanolamine.

Examples of preferred classes of optional acidic pH adjusters are the mineral acids. Nonlimiting examples of mineral acids are hydrochloric acid, nitric acid, phosphoric acid, and sulfuric acid. The identity of the acidic pH adjuster is not limited and any acidic pH adjuster known in the art, alone or in combination, can be used.

An optional alkanolamide to provide composition thickening can be, but is not limited to, cocamide MEA, cocamide DEA, soyamide DEA, lauramide DEA, oleamide MIPA, stearamide MEA, myristamide MEA, lauramide MEA, capramide DEA, ricinoleamide DEA, myristamide DEA, stearamide DEA, oleylamide DEA, tallowamide DEA, lauramide MIPA, tallowamide MEA, isostearamide DEA, isostearamide MEA, and mixtures thereof. Alkanolamides are noncleansing surfactants and are added, if at all, in small amounts to thicken the composition.

The antimicrobial compositions also can contain about 0.01% to about 5%, by weight, and preferably 0.1% to about 3%, by weight, of an optional gelling agent. To achieve the full advantage of the present invention, the antimicrobial compositions contain about 0.25% to about 2.5%, by weight, of a gelling agent. The antimicrobial compositions typically contain a sufficient amount of gelling agent such that the composition is a viscous liquid, gel, paste, semisolid, or solid that can be easily applied to, and rubbed on, the skin of the nose or nasal mucosa, and such that the composition remains in contact with the area of application, i.e., does not run or drip from the nasal passage. Persons skilled in the art are aware of the type and amount of gelling agent to include in the composition to provide the desired composition viscosity or consistency.

The term “gelling agent” as used here and hereafter refers to a compound capable of increasing the viscosity of a water-based composition, or capable of converting a water-based composition to a gel, paste, semisolid, or solid. The gelling agent, therefore, can be organic in nature, for example, a natural gum or a synthetic polymer, or can be inorganic in nature.

The antimicrobial compositions typically are free of a cleansing surfactant and an antimicrobial agent. A cleansing surfactant and antimicrobial agent are not intentionally added to an antimicrobial composition, but may be present in an amount of 0% to about 0.5%, by weight, because a surfactant may be present in a commercial form of a gelling agent to help disperse the gelling agent in water. A surfactant also may be present as an additive or by-product in other composition ingredients. An antimicrobial agent may be present in a composition ingredient as a preservative.

The following are nonlimiting examples of gelling agents that can be used in the antimicrobial composition. In particular, the following compounds, both organic and inorganic, act primarily by thickening or gelling the aqueous portion of the composition:

acacia, agar, algin, alginic acid, ammonium alginate, ammonium chloride, ammonium sulfate, amylopectin, attapulgite, bentonite, C₉₋₁₅ alcohols, calcium acetate, calcium alginate, calcium carrageenan, calcium chloride, caprylic alcohol, carboxymethyl hydroxyethylcellulose, carboxymethyl hydroxypropyl guar, carrageenan, cellulose, cellulose gum, cetearyl alcohol, cetyl alcohol, corn starch, damar, dextrin, dibenzylidine sorbitol, ethylene dihydrogenated tallowamide, ethylene dioleamide, ethylene distearamide, fruit pectin, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, hydroxyethyl stearamide-MIPA, hydroxypropylcellulose, hydroxypropyl guar, hydroxypropyl methylcellulose, isocetyl alcohol, isostearyl alcohol, karaya gum, kelp, lauryl alcohol, locust bean gum, magnesium aluminum silicate, magnesium silicate, magnesium trisilicate, methoxy PEG-22/dodecyl glycol copolymer, methylcellulose, microcrystallinc cellulose, montmorillonite, myristyl alcohol, oat flour, oleyl alcohol, palm kernel alcohol, pectin, PEG-2M, PEG-5M, polyvinyl alcohol, potassium alginate, potassium carrageenan, potassium chloride, potassium sulfate, potato starch, propylene glycol alginate, sodium carboxymethyl dextran, sodium carrageenan, sodium cellulose sulfate, sodium chloride, sodium silicoaluminate, sodium sulfate, stearalkonium bentonite, stearalkonium hectorite, stearyl alcohol, tallow alcohol, TEA-hydrochloride, tragacanth gum, tridecyl alcohol, tromethamine magnesium aluminum silicate, wheat flour, wheat starch, xanthan gum, polyvinylpyrorolidone and derivatives thereof, vinyl ether derivatives (methyl vinyl ether, ethyl vinyl ether, butyl vinyl ether, isobutyl vinyl ether, polymethyl vinyl ether/maleic acid), quaternized vinylpyrrolidone/quaternized dimethylamino ethyl pyrrolidone-based polymers and methacrylate copolymers, vinylcaprolactam/vinylpyrrolidone dimethylamino ethylmethacrylate polymers, vinylpyrrolidone/dimethyl amino ethylmethacrylate copolymers, acid stable and naturally occurring derivatives of guar and modified guar, modified or substituated xanthan, carboxypropyl cellulose, and mixtures thereof.

The following additional nonlimiting examples of gelling agents act primarily by thickening the nonaqueous portion of the composition:

abietyl alcohol, acrylinoleic acid, aluminum behenate, aluminum caprylate, aluminum dilinoleate, aluminum distearate, aluminum isostearates/laurates/palmitates or stearates, aluminum isostearates/myristates, aluminum isostearates/palmitates, aluminum isostearates/stearates, aluminum lanolate, aluminum myristates/palmitates, aluminum stearate, aluminum stearates, aluminum tristearate, beeswax, behenamide, behenyl alcohol, butadiene/acrylonitrile copolymer, a C₂₉₋₇₀ acid, calcium behenate, calcium stearate, candelilla wax, carnauba, ceresin, cholesterol, cholesteryl hydroxystearate, coconut alcohol, copal, diglyceryl stearate malate, dihydroabietyl alcohol, dimethyl lauramine oleate, dodecanedioic acid/cetearyl alcohol/glycol copolymer, erucamide, ethylcellulose, glyceryl triacetyl hydroxystearate, glyceryl triacetyl ricinoleate, glycol dibehenate, glycol dioctanoate, glycol distearate, hexanediol distearate, hydrogenated C₆₋₁₄ olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable glycerides, hydrogenated vegetable oil, hydroxypropylcellulose, isobutylene/isoprene copolymer, isocetyl stearoyl stearate, Japan wax, jojoba wax, lanolin alcohol, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhydride copolymer, octyldodecyl stearoyl stearate, oleamide, oleostearine, ouricury wax, oxidized polyethylene, ozokerite, palm kernel alcohol, paraffin, pentaerythrityl hydrogenated rosinate, pentaerythrityl rosinate, pentaerythrityl tetraabietate, pentaerythrityl tetrabehenate, pentaerythrityl tetraoctanoate, pentaerythrityl tetraoleate, pentaerythrityl tetrastearate, phthalic anhydride/glycerin/glycidyl decanoate copolymer, phthalic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eicosene copolymer, PVP/hexadecene copolymer, rice bran wax, stearalkonium bentonite, stearalkonium hectorite, stearamide, stearamide DEA-distearate, stearamide DIBA-stearate, stearamide MEA-stearate, stearone, stearyl alcohol, stearyl erucamide, stearyl stearate, stearyl stearoyl stearate, synthetic beeswax, synthetic wax, trihydroxystearin, triisononanoin, triisostearin, triisostearyl trilinoleate, trilaurin, trilinoleic acid, trilinolein, trimyristin, triolein, tripalmitin, tristearin, zinc laurate, zinc myristate, zinc neodecanoate, zinc rosinate, zinc stearate, and mixtures thereof.

Exemplary gelling agents useful in the present invention include, but are not limited to,

| | |
|---|---|
| Polyethylene Glycol & Propylene Glycol & Water | (ACULYN 44) |
| Ammonium Acrylatedimethyltaurate/VP Copolymer | (ARISTOFLEX AVC) |
| Glyceryl Stearate & PEG 100 Stearate | (ARLACEL 165) |
| Polyethylene(2)Stearyl Ether | (BRIJ 72) |
| Polyoxyethylene(21)Stearyl Ether | (BRIJ 721) |
| Silica | (CAB-O-SIL) |
| Polyquaternium 10 | (CELQUAT CS230M) |
| Cetyl Alcohol | |
| Cetearyl Alcohol & Cetereth 20 | (COSMOWAX P) |
| Cetearyl Alcohol & Dicetyl Phosphate & Ceteth-10 | (CRODAFOS CES) |
| Phosphate | |
| Ceteth-20 Phosphate & Cetearyl Alcohol & Dicetyl | (CRODAFOS CS-20 Acid) |
| Phosphate | |
| Cetearyl Alcohol & Cetereth 20 | (EMULGADE NI 1000) |
| Sodium Magnesium Silicate | (LAPONITE XLG) |
| Cetyl Alcohol & Stearyl Alcohol & Stearalkonium Chloride | (MACKADET CBC) |
| & Dimethyl Stearamine & Lactic Acid | |
| Cetearyl Alcohol & Stearamidopropyldimethylamine & | (MACKERNIUM Essential) |
| Stearamidopropylalkonium Chloride | |
| Stearalkonium Chloride | (MACKERNIUM SDC-85) |
| Cetearyl Alcohol & Stearamidopropyldimethylamine & | (MACKERNIUM Ultra) |
| Stearamidopropylalkonium Chloride & Silicone Quaternium | |
| 16 | |
| Cetearyl Alcohol & Cetearyl Glucoside | (MONTANOV 68EC) |
| Hydroxyethylcellulose | (NATROSOL 250 HHR CS) |
| Polyquaternium-37 & Mineral Oil & Trideceth-6 | (SALCARE SC 95) |
| Polyquaternium-32 & Mineral Oil & Trideceth-6 | (SALCARE SC 96) |
| Stearic Acid | |
| Cetyl Hydroxyethylcellulose | (NATROSOL Plus 330 CS) |
| Polyvinyl Alcohol, PVP-K30, Propylene Glycol | |
| Stearic Acid, Behenyl Alcohol, Glyceryl Stearate, Lecithin, | (PROLIPID 141) |
| C12-16 Alcohols, Palmic Acid | |
| Beeswax | (saponified beeswax) |
| Beeswax | (synthetic beeswax) |
| Water, Beeswax, Sesame Oil, Lecithin, Methyl paraben | (beesmilk) |
| Polyquaternium 10 | (CELQUAT SC240C) |
| Sodium Acrylate/Sodium Acrylodimethyl Taurate | (SIMULGEL EG) |
| Copolymer & Isohexadecane & Polysorbate 80 | |
| Polyquaternium 44 | (LUVIQUAT Care) |

E. pH

The pH of an antimicrobial composition is less than about 5, and preferably less than about 4.5 at 25° C. To achieve the full advantage of the present invention, the pH is less than about 4. Typically, the pH of a present composition is about 2 to less than about 5, and preferably about 2.5 to about 4.5.

The pH of the composition is sufficiently low such that at least a portion of the organic acid is in the protonated form. The organic acid then has the capability of lowering surface pH, such as skin pH, to provide an effective viral control, without irritating the skin. The organic acid also deposits on the skin and nasal mucosa, and resists removal by rinsing, to provide a persistent antiviral effect.

The present invention is directed to methods of preventing and treating viral infections by administering compositions described herein to the nose and surrounding facial areas, including the nasal cavity. The method involves administration of an antimicrobial composition to the nose and surrounding facial areas, both external and internal, to lower the pH of the treated areas. Generally, a safe and effective amount of the composition is applied to the nose and surrounding facial area. In this context, the term “safe and effective amount” refers to an amount of the composition that provides a therapeutic benefit with minimal or no adverse side effects.

The methods of the present invention include administration of an antimicrobial composition to the nose and surrounding facial areas, wherein the composition is manufactured into final product forms of liquids, sprays, inhalants, pumps, drops, gel, pastes, ointments, dry pastes, wipes, swabs, and like forms for administration to the nose and surrounding facial areas to prevent and treat respiratory tract viral infections.

A preferred method of treating and preventing respiratory tract viral infections involves applying the antimicrobial composition onto the nose and surrounding facial areas, including into the nasal cavity. For antimicrobial compositions in the form of nasal sprays, effective amounts of the composition are sprayed into each nostril one or more times to prevent or treat respiratory tract viral infections. Typically, the spray is administered two to three times into each nostril as an effective method of reducing the transmission of viruses to the respiratory tract, and of preventing and treating respiratory tract viral infections.

When the antimicrobial compositions are administered using product forms such as sprays, pumps, droplets, ointments, gels, pastes, and the like, the compositions generally are prepared by including a gelling agent or a polymeric acid in the composition. The gelling agent and/or polymeric acid enhances the affinity of the organic acids to the skin on and surrounding the nose and the nasal mucosa, and can form a residual continuous or discontinuous film or barrier layer on the treated area of skin, which in turn prolongs the persistent antiviral activity of the composition.

Suitable devices utilized in the administration of the antiviral compositions include those commonly employed, such as liquid containers, droppers, spray containers including pressurized sprayers, pump containers, inhalation devices, atomizers, and the like. The composition also can be incorporated into tissues, wipes, swabs, and similar fibrous and nonfibrous delivery devices for application onto the skin or into the nasal passage by wiping.

To demonstrate the new and unexpected results provided by an application of the antimicrobial compositions to the nose and surrounding facial area, the following examples were prepared, and the ability of the compositions to control Gram positive and Gram negative bacteria, and to control rhinovirus both on the hands and on the nose, was determined. The weight percentage listed in each of the following examples represents the actual, or active, weight amount of each ingredient present in the composition. The compositions are prepared by blending the ingredients, as understood by those skilled in the art and as described below.

The following methods are used in the preparation and testing of the examples:

a) Determination of Rapid Germicidal (Time Kill) Activity of Antibacterial Products. The activity of antibacterial compositions is measured by the time kill method, whereby the survival of challenged organisms exposed to an antibacterial test composition is determined as a function of time. In this test, a diluted aliquot of the composition is brought into contact with a known population of test bacteria for a specified time period at a specified temperature. The test composition is neutralized at the end of the time period, which arrests the antibacterial activity of the composition. The percent or, alternatively, log reduction from the original bacteria population is calculated.

In general, the time kill method is known to those skilled in the art.

The composition can be tested at any concentration up to 100%. The choice of which concentration to use is at the discretion of the investigator, and suitable concentrations are readily determined by those skilled in the art. For example, viscous samples usually are tested at 50% dilution, whereas nonviscous samples are not diluted. The test sample is placed in a sterile 250 ml beaker equipped with a magnetic stirring bar and the sample volume is brought to 100 ml, if needed, with sterile deionized water. All testing is performed in triplicate, the results are combined, and the average log reduction is reported.

The choice of contact time period also is at the discretion of the investigator. Any contact time period can be chosen. Typical contact times range from 15 seconds to 5 minutes, with 30 seconds and 1 minute being typical contact times. The contact temperature also can be any temperature, typically room temperature, or about 25 degrees Celsius.

The bacterial suspension, or test inoculum, is prepared by growing a bacterial culture on any appropriate solid media (e.g., agar). The bacterial population then is washed from the agar with sterile physiological saline and the population of the bacterial suspension is adjusted to about 10⁸ colony forming units per ml (cfu/ml).

The table below lists the test bacterial cultures used in the tests and includes the name of the bacteria, the ATCC (American Type Culture Collection) identification number, and the abbreviation for the name of the organism used hereafter. S. aureus is a Gram positive bacteria, whereas E. coli, K. pneum, and S. choler. are Gram negative bacteria.

| Organism Name | ATCC # | Abbreviation |
|---|---|---|
| Staphylococcus aureus | 6538 | S. aureus |
| Escherichia coli | 11229 | E. coli |
| Klebsiella pneumoniae | 10031 | K. pneum. |
| Salmonella choleraesuis | 10708 | S. choler. |

The beaker containing the test composition is placed in a water bath (if constant temperature is desired), or placed on a magnetic stirrer (if ambient laboratory temperature is desired). The sample then is inoculated with 1.0 ml of the test bacteria suspension. The inoculum is stirred with the test composition for the predetermined contact time. When the contact time expires, 1.0 ml of the test composition/bacteria mixture is transferred into 9.0 ml of Neutralizer Solution. Decimal dilutions to a countable range then are made. The dilutions can differ for different organisms. Selected dilutions are plated in triplicate on TSA+ plates (TSA+ is Trypticase Soy Agar with Lecithin and Polysorbate 80). The plates then are incubated for 24±2 hours, and the colonies are counted for the number of survivors and the calculation of percent or log reduction. The control count (numbers control) is determined by conducting the procedure as described above with the exception that deionized water is used in place of the test composition. The plate counts are converted to cfu/ml for the numbers control and samples, respectively, by standard microbiological methods.

The log reduction is calculated using the formula

$$Log reduction = {log}_{10} ( numbers controlled ) - {log}_{10} ( test sample survivors ) .$$

The following table correlates percent reduction in bacteria population to log reduction:

| % Reduction | Log Reduction |
|---|---|
| 90 | 1 |
| 99 | 2 |
| 99.9 | 3 |
| 99.99 | 4 |
| 99.999 | 5 |

b) Antiviral Residual Efficacy Test

References: S. A. Sattar, Standard Test Method for Determining the Virus-Eliminating Effectiveness of Liquid Hygienic Handwash Agents Using the Fingerpads of Adult Volunteers, Annual Book of ASTM Standards. Designation E1838-96, incorporated herein by reference in its entirety, and referred to as “Sattar I”; and S. A. Sattar et al., Chemical Disinfection to Interrupt Transfer of Rhinovirus Type 14 from Environmental Surfaces to Hands, Applied and Environmental Microbiology, Vol. 59, No. 5, May, 1993, pp. 1579-1585, incorporated herein by reference in its entirety, and referred to as “Sattar II.”

The method used to determine the Antiviral Index of the present invention is a modification of that described in Sattar I, a test for the virucidal activity of liquid hand washes (rinse-off products). The method is modified in this case to provide reliable data for leave-on products.

Modifications of Sattar I include the product being delivered directly to the skin as described below, virus inoculation of the fingerpads as described below, and viral recovery using ten-cycle washing. The inoculated skin site then is completely decontaminated by treating the area with 70% dilution of ethanol in water.

Procedure:

Ten-minute Test:

Subjects (5 per test product) initially wash their hands with a nonmedicated soap, rinse the hands, and allow the hands to dry.

The hands then are treated with 70% ethanol and air dried.

Test product (1.0 ml) is applied to the hands, except for the thumbs, and allowed to dry.

About 10 minutes (±30 seconds) after product application, 10 μl of a Rhinovirus 14 suspension (ATCC VR-284, approximately 1×10⁶ PFU (plaque-forming units)/ml) is topically applied using a micropipette to various sites on the hand within a designated skin surface area known as fingerpads. At this time, a solution of rhinovirus also is applied to the untreated thumb in a similar manner.

After a dry-down period of 7-10 minutes, the virus then is eluted from each of the various skin sites with 1 ml of eluent (Earle's Balanced Salt Solution (EBSS) with 25% Fetal Bovine Serum (FBS)+1% pen-strep-glutamate), washing 10 times per site.

The inoculated skin site then is completely decontaminated by rinsing the area with 70% ethanol. Viral titers are determined using standard techniques, i.e., plaque assays or TCID₅₀ (Tissue Culture Infectious Dose).

One-hour test:

Subjects are allowed to resume normal activities (with the exception of washing their hands) between the 1-hour and 3-hour timepoints. After one hour, a rhinovirus suspension is applied to and eluted from designated sites on the fingerpads exactly as described in above for the 10-minute test.

### EXAMPLE 1

The following compositions were prepared.

| Sample | Composition (by wt %) |
|---|---|
| A | 62% ethanol in water |
| B | 30% ethanol in water |
| C | 2% salicylic acid in 62% ethanol/water |
| D | 2% salicylic acid in 30% ethanol/water |
| E | 2% salicylic acid in dipropylene glycol/water |

The samples were tested for antiviral activity against Rhinovirus 1A and Rotavirus Wa in a time kill suspension test. The following table summarizes the results of the test.

Log 10 Reduction of Virus

| | Rhinovirus 1A | | Rotavirus Wa | |
|---|---|---|---|---|
| Sample | 30 sec | 1 min | 30 sec | 1 min |
| A | <1 log | <1 log | <1 log | <1 log |
| B | <1 log | <1 log | <1 log | <1 log |

| | | |
|---|---|---|
| C | Complete elimination | Complete elimination |
| D | Complete elimination | Complete elimination |
| E | Incomplete inactivation | Incomplete inactivation |

This example illustrates the synergistic antiviral effect provided by the combination of a disinfecting alcohol and an organic acid having a log P of less than one. Samples A and B show that a disinfecting alcohol alone does not provide an acceptable control of viruses. Sample E shows that salicylic acid dissolved in dipropylene glycol and water does not completely inactivate the tested virus serotypes. However, Samples C and D, which are compositions of the present invention, completely eliminate the tested virus serotypes.

### EXAMPLE 2

The following antiviral composition, which is capable of reducing skin pH, was prepared and applied to the fingerpads of human volunteers:

Sample 2

| Material | Percent (by weight) |
|---|---|
| Ethanol | 70.0 |
| Deionized water | 19.8 |
| ULTREZ ® 20¹⁾ | 1.0 |
| Isopropyl Palmitate | 1.0 |
| Mineral oil | 1.0 |
| DC 200 silicone fluid | 1.0 |
| Cetyl alcohol | 1.0 |
| Citric acid | 2.0 |
| Malic acid | 2.0 |
| GERMABEN II²⁾ | 1.0 |
| Triethanolamine | 0.05 |
| | 100.0 |

¹⁾Acrylate/C10-30 Alkyl Acrylate Crosspolymer;
²⁾Preservative containing propylene glycol, diazolidinyl urea, methylparaben, and propylparaben.

The pH of Sample 2 was 3.1.

In the test, Sample 2 was applied to the fingerpads of all fingers, except the thumbs, of eight volunteers. The thumbs were control sites. The volunteers were divided into fours groups of two each. Each group I-IV then was challenged at a predetermined time with rhinovirus titer on all the fingerpads of each hand to determine the time-dependent efficacy of the test composition. At the time appropriate for each group, the skin pH of the fingerpads also was measured to determine the time course of skin pH in response to the test composition. The predetermined test time for rhinoviral challenge and skin pH measurement for each group I-IV were 5 minutes, 1 hour, 2 hours, and 4 hours, respectively. The following table summarizes the average log (rhinoviral titer inoculum), average skin pH, and average log (rhinoviral titer recovered) from the test fingerpads of the volunteers in the study, organized by group.

| | Initial skin | | | |
|---|---|---|---|---|
| | pH after | | Log [Inoculum | Log |
| | application | Skin pH at test | Titer] | [Recovered |
| Group | (average) | time (average) | (average) | Titer] (average) |
| I | 3.0 | 3.0 | 3.9 | 0.23 |
| II | 2.8 | 3.4 | 4.0 | 0.23 |
| III | 3.0 | 3.8 | 3.8 | 0.23 |
| IV | 3.0 | 3.8 | 4.3 | 0.23 |

The data for each group (i.e., different time points) shows that the average recovered rhinoviral titer is less than 1 virus particle, or below the detection limit of the test. This data illustrates the efficacy of the present method after 4 hours and further demonstrates that a skin pH of less than about 4 is effective at completely eliminating a virus challenge. The combination of citric acid, malic acid, and polymeric acid (i.e., ULTREZ® 20) provided a residual barrier layer of organic acids on the fingerpads, which enhanced the persistent antiviral activity of the composition.

### EXAMPLE 3

The clean fingerpads of test subjects were treated with the following compositions. Baseline skin pH readings were measured from the fingerpads prior to treatment with the compositions. Skin pH measurements also were taken immediately after the composition dried on the fingerpads, then again after four hours.

| | | | | | | % |
|---|---|---|---|---|---|---|
| | | Average | Average | Viral | | Hands |
| | | Skin pH | Skin pH | Log 10 | | with |
| Sample | Composition (by wt %) | (T = 0) | (T = 4 hr) | Reduction | | Virus |
| A | 2% citric acid, 2% malic | 2.81 | 3.23 | >3 | log₁₀ | 0 |
| | acid, 62% ETOH, 1.25% | | | | | |
| | hydroxyethylcellulose | | | | | |
| B | 2% citric acid, | 2.64 | 3.03 | >3 | log₁₀ | 0 |
| | 2% tartaric acid, | | | | | |
| | 62% ETOH, 1.25% | | | | | |
| | hydroxyethylcellulose | | | | | |
| C | 2% malic acid, 2% tartaric | 2.66 | 2.94 | >3 | log₁₀ | 0 |
| | acid, 62% ETOH, 1.25% | | | | | |
| | hydroxyethylcellulose | | | | | |
| D | 62% ETOH, 1.25% | 5.53 | 5.13 | <0.5 | log₁₋ | 100 |
| | hydroxyethylcellulose | | | | | |
| E | 2% citric acid, 2% malic | 2.90 | 3.72 | >3 | log₁₀ | 0 |
| | acid, 70% ETOH, 1% | | | | | |
| | polyacrylic acid | | | | | |
| F | 70% ETOH, 1% polyacrylic | 4.80 | 5.16 | 2.0 | log₁₀ | 100 |
| | acid | | | | | |
| G | 70% ETOH, 1.25% | 5.3 | 5.25 | <0.5 | log₁₀ | 100 |
| | hydroxyethylcellulose | | | | | |

¹⁾ETOH is ethanol

Four hours after treatment of the fingerpads with Samples A-G, Rhinovirus 39 at a titer of 1.3×10³ pfu (plaque forming units) was applied to fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 39, i.e., a greater than 3 log reduction, was achieved using the acid-containing compositions containing a mixture of two of citric acid, malic acid, and tartaric acid. The presence of hydroxyethylcellulose or polyacrylic acid assisted in forming a more continuous film or layer of organic acids on the treated fingerpads, which in turn enhanced the persistent antiviral activity of the compositions.

### EXAMPLE 4

### Antibacterial Activity

Log Reduction

| | S. aureus | | E. coli | |
|---|---|---|---|---|
| | ATCC 6538 | | ATCC 11229 | |
| Sample | 30 seconds¹⁾ | 60 seconds¹⁾ | 30 seconds | 60 seconds |
| A | >4.91 | >4.91 | >5.00 | >5.00 |
| B | >4.91 | >4.91 | >5.00 | >5.00 |

¹⁾Contact time on the skin
A. 62% Ethanol, 2% citric acid, 2% malic acid, 1.25% hydroxyethylcellulose
B. 62% Ethanol, 2% citric acid, 2% malic acid, 1.25% hydroxyethylcellulose, and skin emollients

This example illustrates that compositions of the present invention also provide a rapid and broad spectrum antibacterial activity.

### EXAMPLE 5

The clean fingerpads of test subjects were treated with the following composition. Baseline skin pH readings were measured from the fingerpads prior to treatment with the compositions. Skin pH measurements also were taken immediately after the composition dried on the fingerpads.

Immediately after treatment of the fingerpads with the composition, Rhinovirus 14 at a titer of 1.4×10⁴ pfu (plaque forming units) was applied to the fingerpads. The virus was dried on the fingerpads for 10 minutes, then the fingerpads were rinsed with a viral recovery broth containing 75% EBSS and 25% FBS with 1× antibiotics. The sample was diluted serially in viral recovery broth and plated onto H1-HeLa cells. Titers were assayed as per the plaque assay. Complete inactivation of Rhinovirus 14 was achieved with the acid-containing composition resulting in a 4 log reduction.

| | | | Viral Log 10 | |
|---|---|---|---|---|
| | | Solution | Reduction 30 | % Hands |
| Sample | Composition (by wt %) | pH | seconds | with Virus |
| A | 2% citric acid, 2% malic | 3.10 | 4 log | 0 |
| | acid, 70% ETOH, 1% | | | |
| | polyacrylic acid | | | |

### EXAMPLE 6

The following compositions were prepared to test the effect of organic acids and organic acid blends on skin pH and antiviral efficacy.

| | | Average | Average | Viral |
|---|---|---|---|---|
| | | Skin pH | Skin pH | Log10 |
| Sample | Composition (by wt %) | (T = 0) | (T = 2 hr) | Reduction |
| A | 4% citric acid in 70% | 2.97 | 3.64 | >3 log₁₀ |
| | ethanol/water | | | |
| B | 4% malic acid in 70% | 2.91 | 3.94 | >3 log₁₀ |
| | ethanol/water | | | |
| C | 2% citric acid and 2% malic | 2.99 | 3.38 | >3 log₁₀ |
| | acid in 70% ethanol/water | | | |
| D | 4% tartaric acid in 70% | 2.56 | 3.0 | >3 log₁₀ |
| | ethanol/water | | | |

The clean fingerpads of the test subjects were treated with Samples A-D. Baseline skin pH readings were measured from the fingerpads prior to treatment with a composition. Skin pH measurements also were taken immediately after the composition dried on the fingerpads, and again after two hours.

All Samples A-D suppressed skin pH to below 4 for two hours. The combination of citric acid and malic acid (Sample C) maintained a lower pH at two hours than the same acids used singly (Samples A and B). The 4% tartaric acid composition (Sample D) showed the greatest suppression of skin pH.

Two hours after treatment of the fingerpads with the solutions, Rhinovirus 39 at a titer of 4×10⁴ pfu was applied to fingerpads. The virus was dried on the fingerpads for 10 minutes, then the fingerpads were rinsed with a viral recovery broth containing 75% EBSS and 25% FBS with 1× antibiotics. The sample was serially diluted in viral recovery broth and plated onto H1-HeLa cells. Titers were assayed as per the plaque assay. Complete inactivation of Rhinovirus 39 was achieved resulting in a greater than 3 log reduction.

The following examples illustrate that polymeric acids, and especially an acrylic acid homopolymer or copolymer, in the presence of alcohol impart antiviral efficacy. The polymeric acids have a low pH and good substantivity to skin, which effectively maintains a low skin pH over time, and helps provide a persistent antiviral efficacy. The polymeric acids also help provide an essentially continuous layer or film of an organic acid on treated surfaces, which in turn enhances the persistent antiviral activity of the composition.

A synergistic effect on the lowering of skin pH was demonstrated with using acrylic acid-based polymer in the presence of alcohol. However, an acrylic acid-based polymer in the absence of an alcohol did not maintain a reduced skin pH to the same degree over time. Importantly, skin pH reduction is less dependent on composition pH when a polymeric acid is used in conjunction with an alcohol. The synergy demonstrated between the polymeric acid and the alcohol was unexpected and is a novel way of providing the lowered skin pH that provides a desired antiviral efficacy.

A synergistic effect on a rapid and persistent antiviral activity also is demonstrated when an acrylic acid-based polymer is used in conjunction with polycarboxylic acids. It has been found that utilizing a low amount of a polymeric acid (e.g., about 0.1% to about 2%, by weight) together with a polycarboxylic acid, like citric acid, malic acid, tartaric acid, and mixtures thereof, enhances the antiviral activities of the polycarboxylic acids. This synergistic effect allows a reduction in the polycarboxylic acid concentration in an antiviral composition, without a concomitant decrease in antiviral efficacy. This reduction in polycarboxylic acid concentration improves composition mildness by reducing the irritation potential of the composition. It is theorized, but not relied upon herein, that the polymeric acid assists in forming a residual barrier film or layer of organic acids on a treated surface, which enhance the persistent antiviral activity of the composition.

### EXAMPLE 7

A composition containing a polyacrylic acid (1% by wt), i.e., ULTREZ 20, available from Novean Europe, was prepared in 70% aqueous ethanol and in water. Each composition (1.8 ml) was applied to the thumb, index, and middle fingers of a test subject. Skin pH readings were measured prior to treatment (baseline), immediately after the fingers were dry, and again after two hours. The average skin pH readings are summarized below.

| | | | | Viral |
|---|---|---|---|---|
| | Average skin pH | | | log 10 |
| | Baseline | T = 0 | T = 2 hrs. | reduction |
| 70% ethanol | 5.65 | 5.3 | 5.2 | <0.2 |
| Polyacrylic acid (1%) (70% | 5.63 | 4.4 | 4.5 | 1.8 |
| aqueous ethanol) | | | | |
| Polyacrylic acid (1%) (water) | 5.64 | 4.5 | 4.7 | 1.5 |

The polyacrylic acid suppressed skin pH to about 4.5 initially, and skin pH remains under 5 after two hours. The composition with ethanol suppressed skin pH slightly lower (4.4) than the composition free of ethanol (4.5). This result suggests a synergistic effect on lowering skin pH when a polyacrylic acid is applied with ethanol.

Two hours after treatment of the fingerpads with the above compositions, Rhinovirus 39 was applied to the fingerpads that had been treated at a titer of 9.8×10² pfu. The virus was dried on the fingerpads for 10 minutes, then the fingerpads were rinsed with viral recovery broth. The broth was serially diluted in viral recovery broth and plated onto H1-HeLa cells. Titers were assayed as per the plaque assay. Both compositions reduced the viral titer. However, the composition containing ethanol exhibited slightly greater efficacy against Rhinovirus by reducing the titer by 1.8 log versus 1.5 log for the composition without ethanol.

This data illustrates that polyacrylic acid suppresses skin pH resulting in antiviral efficacy. The data also illustrates that polyacrylic acid and ethanol act synergistically to lower skin pH, thus resulting in a greater efficacy against rhinovirus.

To demonstrate this efficacy, the following eight compositions were prepared, wherein solutions containing a polyacrylic acid (with and without ethanol) were buffered to a pH of about 4.5, 5.0, 5.5, or 6.0.

| | | | Avg. | Viral |
|---|---|---|---|---|
| | | Solution | Skin | Log₁₀ |
| Sample | Composition (by wt %) | pH | Ph 2 hrs. | Reduction |
| A | 1% ULTREZ 20/70% ethanol | 4.54 | 4.52 | >2 log₁₀ |
| B | 1% ULTREZ 20/70% ethanol | 5.10 | 4.87 | >2 log₁₀ |
| C | 1% ULTREZ 20/70% ethanol | 5.54 | 4.41 | >2 log₁₀ |
| D | 1% ULTREZ 20/70% ethanol | 6.17 | 4.32 | >2 log₁₀ |
| E | 1% ULTREZ 20 | 4.57 | 4.93 | <1 log₁₀ |
| F | 1% ULTREZ 20 | 5.12 | 5.46 | <1 log₁₀ |
| G | 1% ULTREZ 20 | 5.55 | 5.33 | <1 log₁₀ |
| H | 1% ULTREZ 20 | 6.32 | 5.70 | <1 log₁₀ |

The effect of the eight compositions on both skin pH and viral efficacy was tested. Each composition (1.8 ml) was applied to the thumb, index, and middle fingers of a test subject. Skin pH readings were measured prior to treatment (baseline), immediately after the product had dried, and again after two hours.

The skin pH data indicate that a polyacrylic acid and ethanol function synergistically to suppress skin pH because each composition containing ethanol in combination with the polyacrylic acid suppressed skin pH to a lower value than compositions free of ethanol. Compositions containing ethanol and polyacrylic acid lowered skin pH to between pH 4 and 5 independent of the solution pH. In contrast, compositions free of ethanol suppress the skin pH only to between pH 5-6 and the final skin pH is similar to the solution pH.

To test the viral efficacy of the above compositions, Rhinovirus 39 at a titer of 1.7×10³ pfu was applied to the fingerpads after two hours. The virus dried for 10 minutes, eluted and diluted serially in viral recovery broth. Samples were plated on H1-HeLa cells, and virus titer was assayed as per the plaque assay method. The compositions containing ethanol in combination with polyacrylic acid had a greater than 2 log reduction in viral titers, whereas compositions free of ethanol exhibited a less than 1 log reduction in viral titers. Therefore, a synergism exists between polyacrylic acid and ethanol in reducing skin pH, which provides greater antiviral efficacy against rhinovirus. It is theorized, but not relied upon herein, that the ethanol helps provide a more continuous film or layer of the organic acid on the skin, for example, by reducing the surface tension of the composition for a more even and uniform application of the composition to a surface, and particularly skin.

### EXAMPLE 8

The following compositions were prepared to further illustrate the antiviral efficacy provided by a polyacrylic acid.

| | | | Avg. | % |
|---|---|---|---|---|
| | | | Skin | Hands |
| | | Solution | pH | with |
| Sample | Composition (by wt %) Thickeners | pH | 2 hrs. | Virus |
| A | 1% polyacrylic acid | 4.21 | 4.7 | 63% |
| B | 5.5% CRODAFOS Acid¹⁾ | 5.41 | 5.0 | 100% |
| C | 1.25% NATROSOL 250 HHR CS²⁾ | 6.32 | 5.3 | 100% |

¹⁾CRODAFOS CS20 Acid is Ceteth-20 & Cetaryl Alcohol & Dicetyl Phosphate; and
²⁾NATROSOL 250 HHR CS is hydroxyethylcellulose.

Samples A-C (1.8 ml) were applied to the thumb, index, and middle fingers of clean hands. Skin pH readings were taken prior to treatment (baseline), immediately after the fingers were dry, and again after two hours for Samples A and B and after four hours for Sample C. The averages of the skin pH values are provided in the above table.

Sample A containing polyacrylic acid lowered the skin pH to the greatest extent with a final skin pH after two hours of pH 4.7. Neither Sample B nor Sample C lowered the skin pH below pH 5.0. This data indicates that polyacrylic acid has an ability to suppress skin pH and maintain a low skin pH for a least two hours.

The viral efficacy of Samples A-C against Rhinovirus 39 was also tested. A viral load of about 10³ pfu was spread over the thumb, index, and middle fingers of each treated hand and allowed to dry for 10 minutes. The fingers then were rinsed with viral recovery broth and samples were serially diluted and plated on H1-HeLa cells. Viral titers were measured using the plaque assay. For both Samples B and C, 100% of the hands were positive for rhinovirus, which indicates little efficacy of these compositions against rhinovirus. In contrast, Sample A demonstrated a viral efficacy because only 63% of the hands were found positive for rhinovirus.

### EXAMPLE 9

Example 7 demonstrated that a synergism exists between polyacrylic acid and ethanol, which results in suppression of skin pH and antiviral efficacy. The following compositions were prepared to examine the effectiveness of polycarboxylic acid blends and a single polycarboxylic acid composition, each in combination with polyacrylic acid and ethanol, on antiviral efficacy. A preferred antiviral composition contains the least amount of organic acid required to demonstrate a persistent antiviral efficacy.

The compositions were applied to the fingerpads of clean hands. After the indicated times, about 10³ to 10⁴ pfu of Rhinovirus 39 was applied to the hands and allowed to dry for 10 minutes. The virus was recovered by rinsing the hands with viral recovery broth. The samples then were diluted serially in viral recovery broth and plated on H1-HeLa cells. Viral titers were determined by plaque assay. The percentage of hands that were positive for rhinovirus is summarized below.

| | | | % of |
|---|---|---|---|
| | | | Hands Positive |
| Composition (by wt %) | Time | | for Rhinovirus |
| 70% ethanol | 15 | min. | 100% |
| 1% citric acid/1% malic acid/10% | 1 | hr. | 100% |
| ethanol/water | | | |
| 1% polyacrylic acid/4% citric acid/70% | 4 | hrs. | 91% |
| ethanol/water | | | |
| 1% polyacrylic acid/1% citric acid/1% malic | 4 | hrs. | 0% |
| acid/70% ethanol/water | | | |

A composition containing 70% ethanol alone was not effective as an antiviral composition. Citric acid (1%) and malic acid (1%) lost effectiveness against rhinovirus after one hour because 100% of the hands were found to be positive for rhinovirus. In contrast, when a composition containing 1% citric and 1% malic acids are applied to the hands in combination with polyacrylic acid and 70% ethanol, no virus was detected on the hands after four hours. A single acid (4% citric acid) in combination with a polyacrylic acid and ethanol was less effective against rhinovirus because 91% of hands were found to be positive for rhinovirus after four hours.

This data demonstrates that using a polyacrylic acid and ethanol allows the use of a lower concentration of polycarboxylic acid to achieve a desired antiviral efficacy. This improvement is attributed, at least in part, to forming a residual film or layer of the organic acids on the skin.

### EXAMPLE 10

The use of a polyacrylic acid and ethanol in a composition suppresses skin pH to a value below the solution pH, as demonstrated in Example 7. To test whether antiviral compositions containing citric acid, malic acid, polyacrylic acid, and ethanol can be buffered to a higher solution pH and still provide a skin pH at or below pH 4 to obtain a persistent antiviral activity, the following compositions were prepared.

| | | | Skin | Skin | |
|---|---|---|---|---|---|
| | | Solution | pH | pH | Viral |
| Sample | Composition (by wt %) | pH | Initial | 4 hrs. | Reduction |
| A | 1% ULTREZ 20/2% citric | 3.2 | 2.9 | 3.7 | >3 log₁₀ |
| | acid/2% malic acid/70% | | | | |
| | ethanol | | | | |
| B | 1% ULTREZ 20/2% citric | 4.34 | 3.4 | 3.7 | >3 log₁₀ |
| | acid/2% malic acid/70% | | | | |
| | ethanol | | | | |
| C | 1% ULTREZ 20/2% citric | 4.65 | 3.6 | 3.8 | >3 log₁₀ |
| | acid/2% malic acid/70% | | | | |
| | ethanol | | | | |

The compositions (1.8 mL) were applied to the thumb, index, and middle fingers of clean hands. Skin pH readings were measured prior to treatment (baseline), immediately after the fingers were dry, and again after four hours. The average of the skin pH values are plotted above.

Initial skin pH of skin treated with Samples A-C were suppressed to between pH 2.9 and 3.6, wherein the lower the solution pH, the lower the initial skin pH. However, after four hours, the skin pH for all three compositions was about pH 3.7. Consistent with previous examples, solution pH did not predict subsequent skin pH.

The viral efficacy of Samples A-C against Rhinovirus 39 also was tested. A viral load of about 10³ pfu was spread over the thumb, index, and middle fingers of each treated hand and allowed to dry for 10 minutes. The fingers then were rinsed with viral recovery broth and samples were diluted serially and plated on H1-HeLa cells. Viral titers were measured using the plaque assay. No virus was recovered from any of the hands indicating that all three Samples A-C have antiviral efficacy.

This data demonstrates that when citric acid and malic acid are utilized in a composition in combination with a polyacrylic acid and ethanol, the pH of the solution can be buffered to a higher, e.g., milder and safer, pH for application to the skin, while still retaining an ability to suppress skin pH and exhibit antiviral activity. This result also is attributed, at least in part, to the residual layer or film of organic acid that remains on the skin after evaporation of volatile composition ingredients.

The following tests demonstrate that a composition of the present invention provides an essentially continuous barrier layer of organic acid on a treated surface. In particular, the following tests show that a present composition resists rinsing from a treated surface, e.g., at least 50% of the nonvolatile composition ingredients (including the organic acid) remains on a treated surface after three rinsings, as determined from NMR and IR spectra. In addition, an effective antiviral amount of the nonvolatile composition ingredients remains on a treated surface after 10 rinsings, also determined using NMR and IR spectra.

In the following tests, an aqueous composition containing, by weight, 2% malic acid, 2% citric acid, 1% polyacrylic acid, 62% ethanol, and 0.5% hydroxyethylcellulose as a gelling agent (Composition A) was compared to an aqueous composition, containing 2% malic acid, 2% citric acid, and 62% ethanol (Composition B). The compositions were applied to a glass surface to provide a film. From infrared (IR) and nuclear magnetic resonance (NMR) spectra of the film taken after each rinse, it was determined that Composition B was completely rinsed from the surface after one rinsing with water. Composition B therefore failed to exhibit water resistance and failed to provide a film or layer of nonvolatile composition ingredients on the surface.

In contrast, IR and NMR spectra showed that Composition A provided a rinse-resistant film or layer of composition ingredients on the treated surface. The amount of composition ingredients that remained on the treated surface was reduced over the first three rinsings, then resisted further removal from the treated surface in subsequent rinses. The IR and NMR spectra showed that detectable and effective amounts of the nonvolatile composition ingredients remained on the treated surface after 10 water rinses.

Another test was performed to measure the contact angle of water on a surface. “Contact angle” is a measure of the wetting ability of water on a surface. In this test, Compositions A and B were applied to a glass surface and allowed to dry. Contact angle then was measured for glass treated with Compositions A and B, both unrinsed and rinsed, using deionized water. The contact angle of bare, i.e., untreated, glass also was measured as a control. The following table summarizes the results of the contact angle test.

| | Compo- | Compo- | | | |
|---|---|---|---|---|---|
| | sition A | sition A | Composition B | Composition B | Bare |
| | Unrinsed | Rinsed | Unrinsed | Rinsed | Glass |
| Avg | 45.96 | 72.66 | 6.69 | 41.51 | 38.47 |
| Reading | | | | | |
| (degrees) | | | | | |
| Change in | | 26.7 | | 34.8 | |
| degrees | | | | | |
| % Change | | 58.1 | | 520.2 | |

The contact angle data shows that Composition A modifies the glass surface and provides a persistent barrier film or layer on the glass surface. The data also shows that Composition B is rinsed from the surface because the contact angle after rinsing of Composition B is essentially the same as that of bare glass.

Another test was performed to demonstrate metal ion uptake by a residual film of Composition A. In this test, films of Composition A were formed on glass, dried at least 4 hours, then exposed to solutions having a 0.5 M concentration of metal ions. Samples then were analyzed by SEM scan. The data in the following table shows that a film resulting from Composition A effectively binds several types of metal ions. It is theorized, but not relied upon, that this is a surface phenomenon because no mechanism for transporting metal ions into the film is known.

Composition A Films on Glass (Metal-Soaked & Deionized Water Rinsed)
(unless otherwise specified)

| Soaking Solution | EDS atomic % | EDS wt % |
|---|---|---|
| 0.56 wt % CaCl₂ in formula | 0.63% Ca | 1.71% Ca |
| on 316 SS-No Rinse | | |
| 0.1 M Ca on 316 SS | 0.13% Ca | 0.21% Ca |
| 0.5 M Ca on 316 SS | 0.34% Ca | 0.54% Ca |
| 0.5 M Ca w/ more rinsing | 0.07% Ca | 0.12% Ca |
| on 316 SS | | |
| 0.5 M Cu on 316 SS | 0.65% Cu | 1.59% Cu |
| 0.5 M Fe on Al 6061 | 0.41% Fe | 1.14% Fe |
| 0.5 M Zn on Al 6061 | 0.24% Zn | 0.90% Zn |
| Metal Coupon analysis | | |
| DI water on 316 SS | 0% Ca, 0% Cu, 0% Zn | 0% Ca, 0% Cu, |
| Fe compensated for in | | 0% Zn |
| above datum | | |
| DI water on Al 6061 | 0.07% Ca, 0.08% Fe, | 0.18% Ca, 0.29% |
| | 0.03% Cu [from Al] | Fe, 0.11% |
| | | Cu [from Al] |

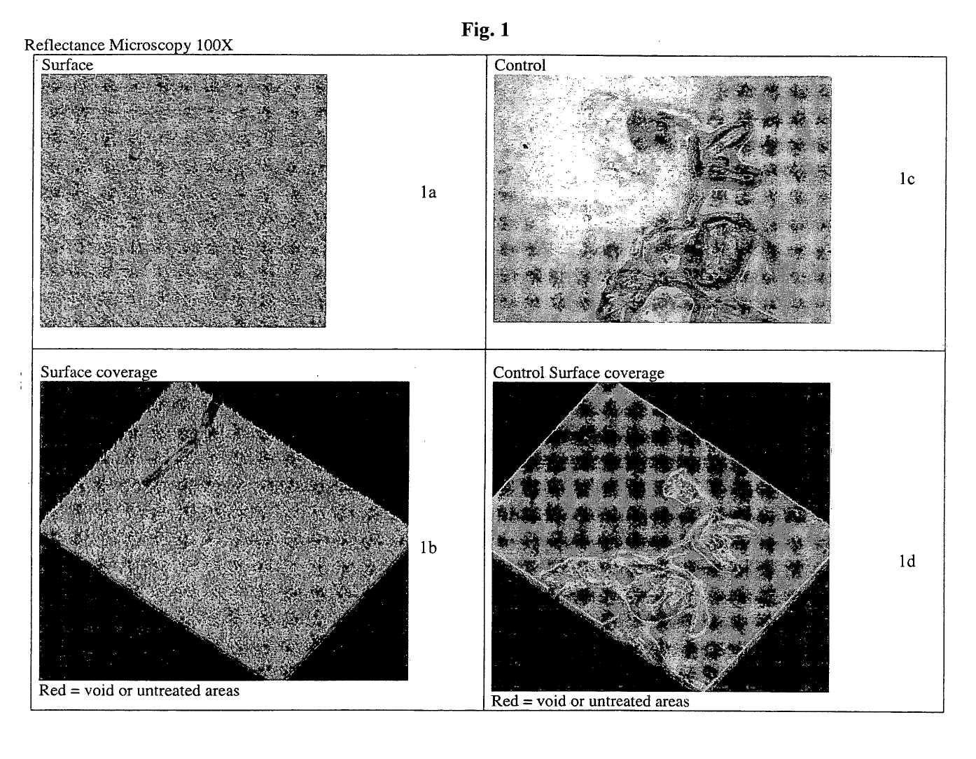

Reflectance micrographs showing the surface coverage of Compositions A and B also were taken (FIG. 1). The attached micrographs show that Composition A provides an essentially complete surface coverage, i.e., a more even coverage of Composition A on a treated surface, which provides an essentially continuous layer or film of nonvolatile composition ingredients on the surface. The attached micrographs are a digital conversion of reflectance values, which provide a direct correlation to surface coverage. The micrographs demonstrate that Composition A (FIGS. 1a) and 1b)) provides a film having improved adhesion, dispersion, and crystal formation compared to Composition B (FIGS. 1c) and 1d)).

A rhinovirus infection typically is the result of contact between a contaminated finger and the nose or eye of an individual. Such contact provides the virus a route of entry to the upper respiratory tract and a respiratory infection ensues. The previous examples illustrate that the present compositions provide effective and persistent antiviral efficacy when applied to the hands. However, the normal course of a day soils the hands and provides opportunities for the antiviral efficacy of the composition on the hands to be compromised. In such an event, or if the individual does not use an antiviral composition on the hands either regularly or at all, the fingers can be contaminated with a virus, which in turn can lead to a respiratory infection after the nose or eye is contacted by the contaminated finger.

By applying a present composition to internal and/or external areas of the nose, and to surrounding facial skin, a persistent antiviral activity is imparted to the treated areas. These skin areas are not subjected to the same conditions as the hands in the normal course of a day, such that the persistent antiviral activity will remain on the treated areas of the nose, even if the treated fingers become contaminated. Therefore, application of the antimicrobial compositions to the nose and surrounding facial areas provides a second level of persistent antiviral activity.

### EXAMPLE 11

A time kill test was performed on additional bacteria and a fungus to demonstrate the broad spectrum efficacy of a composition of the present invention. In this test, the following antimicrobial composition was tested.

| Ingredient | Weight Percent |
|---|---|
| Cetyl Alcohol | 1.00 |
| Glycerin | 1.00 |
| Isopropyl Palmitate | 1.00 |
| Dimethicone 100 CST | 1.02 |
| Ethanol SDA-40B | 3.09 |
| Natrosol 250 HHX | 0.26 |
| Deionized Water | 10.94 |
| Deionized Water | 17.65 |
| ULTREZ 10 Polymer | 1.01 |
| Ethanol SDA-40B | 58.82 |
| Citric Acid | 2.00 |
| Malic Acid | 2.00 |
| Sodium Hydroxide 50% | 0.22 |

The above-composition was tested for an ability to control the following microorganisms under the following conditions:

| | |
|---|---|
| Test Systems: | Staphylococcus aureus ATCC 6538 |
| | Escherichia coli ATCC 11229 |
| | Listeria monocytogenes ATCC 7644 |
| | Enterobacter cloacae ATCC 13047 |
| | Candida albicans ATCC 10231 |
| Test Temperature: | Ambient (20-25° C.) |
| Exposure Time: | 15 and 30 seconds |
| Neutralizer | 99 mL of D/E Broth |
| | A neutralizer screen performed as part of the testing |
| | verified that the neutralizer adequately neutralized |
| | the products and was not detrimental to the tested |
| | organisms. |
| Subculture | D/E Agar |
| Medium: | |
| Incubation: | 35 ± 2° C. for 48 ± 4 hours |
| | (for S. aureus, E. coli, L. monocytogenes) |
| | 30 ± 2° C. for 48 ± 4 hours (for E. cloacae) |
| | 26 ± 2° C. for 72 ± 4 hours (for C. albicans) |

The Test Data Summarized are Below

### Inoculum Numbers (CFU/mL)

| Test System | A | B | Average |
|---|---|---|---|
| Staphylococcus aureus ATCC 6538 | 30 × 10⁶ | 29 × 10⁶ | 3.0 × 10⁷ |
| Escherichia coli ATCC 11229 | 18 × 10⁶ | 18 × 10⁶ | 1.8 × 10⁷ |
| Listeria monocytogenes ATCC 13047 | 26 × 10⁶ | 29 × 10⁶ | 2.8 × 10⁷ |
| Enterobacter cloacae ATCC 13047 | 31 × 10⁶ | 35 × 10⁶ | 3.3 × 10⁷ |
| Candida albicans ATCC 10231 | 24 × 10⁵ | 26 × 10⁵ | 2.5 × 10⁶ |

### Staphylococcus aureus ATCC 15442

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | <100, <100 | <100 | >5.48 | >99.999 |
| 30 | <100, <100 | <100 | >5.48 | >99.999 |

### Escherichia coli ATCC 11229

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | 2 × 10², <100 | <1.5 × 10² | >5.08 | >99.999 |
| 30 | <100, <100 | <100 | >5.26 | >99.999 |

### Listeria monocytogenes ATCC 7644

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | <100, 3 × 10² | <2.0 × 10² | >5.15 | >99.999 |
| 30 | <100, <100 | <100 | >5.45 | >99.999 |

### Enterobacter cloacae ATCC 13027

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | <100, | <100 | >5.52 | >99.999 |
| | contamination | | | |
| 30 | 5 × 10², 6 × 10² | 5.5 × 10² | 4.78 | >99.998 |

### Candida albicans ATCC 10231

| Exposure | | Average | | |
|---|---|---|---|---|
| Time | Survivors | Survivors | Log | Percent |
| (Seconds) | (CFU/mL) | (CFU/mL) | Reduction | Reduction |
| 15 | <100, <100 | <100 | >4.40 | >99.996 |
| 30 | <100, <100 | <100 | >4.40 | >99.996 |

The data shows that a composition of present invention exhibits about a 4 to 5 log reduction at 15 and 30 seconds of exposure time against Staphylococcus aureus ATCC 6538, Escherichia coli ATCC 11229, Listeria monocytogenes ATCC 7644, Enterobacter cloacae ATCC 13047, and Candida albicans ATCC 10231.

The above data shows that a present antimicrobial composition also is effective in controlling fungi, including yeasts and molds. Fungi control is important because fungi can cause a number of plant and animal diseases. For example, in humans, fungi cause ringworm, athlete's foot, and several additional serious diseases. Because fungi are more chemically and genetically similar to animals than other organisms, fungal diseases are very difficult to treat. Accordingly, prevention of fungal disease is desired. The prototype activity against fungi was examined using the yeast Candida albicans. The genus Candida contains a number of species, however, Candida albicans was tested because it is the most frequent cause in candidiasis. Candida albicans can be found in the alimentary tract, mouth, and vaginal area, and can cause diseases including oral candidiasis, also called thrush, vaginitis, alimentary candidiasis, and cutaneous and systemic candidiasis. In particular, a present invention is efficacious in controlling yeasts, such as Candida albicans, demonstrating a log reduction of at least 4 after a 15 second exposure time to a present antimicrobial composition.

The following example illustrates the effectiveness of applying a present antimicrobial composition to the nose and surrounding facial areas.

### EXAMPLE 12

The baseline skin pH of the infratip lobule of each test subject was measured. The nose of each test subject then was treated with the composition of Example 2 (0.1 mL), and the skin pH was measured immediately, after 30 minutes, after two hours, and after four hours for one subject. The skin pH remained below 4.0 for up to two hours after treatment and below 4.2 after four hours.

| | Infratip Lobule Skin pH | | | | |
|---|---|---|---|---|---|
| Test | | Time after Treatment | | | |
| Subject | Baseline | T = 0 | T = 30 min. | T = 2 hrs. | T = 4 hrs. |
| 1 | 6.01 | 3.78 | 4.00 | 4.22 | N/A |
| 2 | 6.11 | 3.36 | 3.47 | 3.96 | N/A |
| 3 | 5.72 | 3.28 | 3.33 | N/A | 4.17 |
| 4 | 5.36 | 3.51 | 3.65 | 3.77 | N/A |
| 5 | 5.04 | 2.86 | 3.07 | 3.65 | N/A |
| Average | 5.65 | 3.36 | 3.50 | 3.90 | 4.17 |

The present invention, therefore, encompasses applying an effective amount of an antimicrobial composition onto the nose and surrounding facial areas, including the nasal passages, to impart a persistent antiviral activity to the treated surface. The presence of the compositions helps retard the transmission of a virus from a contaminated hand or finger that contacts the nose or nasal cavity.

An antimicrobial composition of the present invention can be formulated into a variety of product forms, including liquids, gels, ointments, pastes, dry pastes, or semisolids, The liquid forms can be a solution, dispersion, emulsion, or similar product form. Gel and semisolid product forms can be transparent or opaque, designed for application by stick dispersion or by the fingers, for example. The present antimicrobial composition can be manufactured as dilute ready-to-use compositions, or as concentrates that are diluted prior to use.

Yet another product form is an incorporation of the composition into a web material or a swab. The composition then can be applied to the skin by wiping nose and surrounding facial areas, including the nasal passages, with the web material containing the composition.

In one embodiment of the present invention, a person who either (a) is suffering from a rhinovirus cold, or is likely to be exposed to other individuals suffering from rhinovirus colds, or (b) is suffering from a rotaviral infection, or is likely to be exposed to other individuals suffering from a rotaviral infection, can apply an antimicrobial composition to his or her nose and surrounding facial areas, including the nasal passages. This application kills bacteria and inactivates rhinovirus, rotavirus, and other nonenveloped virus particles present on the treated areas or later contaminated by the virus. The applied composition provides a persistent antiviral activity, and preferably forms a persistent continuous or discontinuous film or barrier layer on the treated surfaces. Nonenveloped viruses, like rhinovirus and rotavirus particles, therefore, are not transmitted to noninfected individuals via hand-to-nose or finger-to-nose transmission. The amount of the composition applied, the frequency of application, and the period of use will vary depending upon the level of disinfection desired, e.g., the degree of microbial contamination.

The present antimicrobial compositions provide the advantages of a broad spectrum kill of Gram positive and Gram negative bacteria, and a virus control, in short contact times. The composition preferably imparts a persistent antiviral activity to the skin and mucosa in the nasal area of the face.

The antimicrobial compositions impart a persistent antiviral activity to the treated external and internal areas of the nose and provide the advantages of a broad spectrum kill of Gram positive and Gram negative bacteria, and a broad spectrum viral control, in short contact times. The present compositions are effective in short contact time because of the reduced pH of the composition, the synergistic effect provided by the combination of a disinfecting alcohol and an organic acid, a persistent activity is enhanced because of a residual barrier layer or film of composition ingredients that can remain on the skin after evaporation of the volatile components of the composition.

Obviously, many modifications and variations of the invention as hereinbefore set forth can be made without departing from the spirit and scope thereof, and, therefore, only such limitations should be imposed as are indicated by the appended claims.

What is claimed is:

1. A method of preventing and treating a respiratory tract virus infection comprising applying a composition to one or more of a nose, surrounding facial skin, and nasal passages of an individual, said composition comprising:
(a) about 25% to about 75%, by weight of a disinfecting alcohol;
(b) a virucidally effective amount of an organic acid; and
(c) water,
wherein the composition has a pH of about 5 or less at 25° C.

2. The method of claim 1 wherein the composition is allowed to remain on the nose, surrounding facial skin, and nasal passages, and dry.

3. The method of claim 1 wherein the virus is an acid-labile virus.

4. The method of claim 1 wherein the acid-labile virus comprises a rhinovirus serotype.

5. The method of claim 1 wherein the composition lowers a pH of the nose, surrounding facial skin, and nasal passages treated by the composition to less than 4 after drying.

6. The method of claim 1 wherein the composition forms a barrier layer comprising the organic acid on the nose and surrounding facial skin.

7. The method of claim 1 wherein an essentially continuous layer comprising the organic acid is formed on the nose and surrounding facial skin.

8. The method of claim 1 wherein the composition imparts a persistent antiviral activity to the treated nose, surrounding facial skin, and nasal passages.

9. The method of claim 1 wherein the disinfecting alcohol is present in the composition in an amount of about 30% to about 75%, by weight of the composition.

10. The method of claim 1 wherein the disinfecting alcohol comprises one or more C₁₋₆ alcohol.

11. The method of claim 10 wherein the disinfecting alcohol is selected from the group consisting of methanol, ethanol, isopropyl alcohol, n-butanol, n-propyl alcohol, and mixtures thereof.

12. The method of claim 1 wherein the composition comprises about 0.05% to about 15%, by weight, of an organic acid.

13. The method of claim 1 wherein the organic acid in the composition has a log P of less than one.

14. The method of claim 1 wherein the organic acid in the composition has a log P of one or greater.

15. The method of claim 1 wherein the organic acid comprises a first organic acid having a log P of less than one and a second organic acid having a log P of one or greater.

16. The method of claim 1 wherein the organic acid has a water solubility of at least about 0.05% by weight, at 25° C.

17. The method of claim 1 wherein the organic acid comprises one or more of a monocarboxylic acid, a polycarboxylic acid, a polymeric acid having a plurality of carboxylic, phosphate, sulfonate, and/or sulfate moieties, anhydrides thereof, or mixtures thereof.

18. The method of claim 1 wherein the organic acid comprises a monocarboxylic acid having a structure RCO₂H, wherein R is C₁₋₆alkyl, hydroxyC₁₋₆alkyl, haloC₁₋₆alkyl, phenyl, or substituted phenyl.

19. The method of claim 18 wherein the monocarboxylic acid is selected from the group consisting of acetic acid, propionic acid, hydroxyacetic acid, lactic acid, benzoic acid, phenylacetic acid, phenoxyacetic acid, zimanic acid, 2-, 3-, or 4-hydroxybenzoic acid, anilic acid, o-, m-, or p-chlorophenylacetic acid, o-, m-, or p-chlorophenoxyacetic acid, and mixtures thereof.

20. The method of claim 1 wherein organic acid comprises a polycarboxylic acid containing two to four carboxylic acid groups, and optionally contains one or more hydroxyl group, amino group, or both.

21. The method of claim 20 wherein the polycarboxylic acid is selected from the group consisting of malonic acid, succinic acid, glutaric acid, adipic acid, pimelic acid, suberic acid, azelaic acid, sebacic acid, fumaric acid, maleic acid, tartaric acid, malic acid, maleic acid, citric acid, aconitic acid, and mixtures thereof.

22. The method of claim 20 wherein the organic acid comprises an anhydride of a polycarboxylic acid.

23. The method of claim 1 wherein the organic acid comprises a polymeric acid having a molecular weight of about 500 to about 10,000,000 g/mol.

24. The method of claim 23 wherein the polymeric acid is water soluble or water dispersible.

25. The method of claim 23 wherein the polymeric acid is selected from the group consisting of a polymeric carboxylic acid, a polymeric sulfonic acid, a sulfated polymer, a polymeric phosphoric acid, and mixtures thereof.

26. The method of claim 23 wherein the polymeric acid comprises a homopolymer or a copolymer of acrylic acid.

27. The method of claim 1 wherein the organic acid comprises a polycarboxylic acid and a polymeric carboxylic acid.

28. The method of claim 27 wherein the polycarboxylic acid comprises citric acid, malic acid, tartaric acid, and mixtures thereof, and the polymeric carboxylic acid comprises a homopolymer or a copolymer of acrylic acid or methacrylic acid.

29. The method of claim 28 wherein the polymeric carboxylic acid comprises a homopolymer or a copolymer of acrylic acid.

30. The method of claim 27 wherein the composition further comprises a gelling agent.

31. The method of claim 1 wherein the composition has a pH of about 2 to less than about 5.

32. The method of claim 5 wherein the treated skin has a pH of less than 4 two hours after application of the composition.

33. The method of claim 1 wherein the composition further comprises about 0.1% to about 30% of a polyhydric solvent selected from the group consisting of a diol, a triol, and mixtures thereof.

34. The method of claim 1 wherein the composition further comprises about 0.1% to about 30%, by weight, of a hydrotrope.

35. The method of claim 1 wherein the composition further comprises about 0.1% to about 3%, by weight, of a gelling agent.

36. The method of claim 35 wherein the gelling agent comprises a natural gum, a synthetic polymer, a clay, an oil, a wax, or mixtures thereof.

37. The method of claim 35 wherein the gelling agent is selected from the group consisting of cellulose, a cellulose derivative, guar, a guar derivative, algin, an algin derivative, a water-insoluble C₈-C₂₀ alcohol, carrageenan, a smectite clay, a polyquaternium compound, and mixtures thereof.

38. The method of claim 1 wherein the composition imparts a log reduction of at least 3 against a nonenveloped virus on the treated skin surface of at least about four hours after contact.

39. The method of claim 1 wherein the composition imparts a log reduction of at least 2 against a nonenveloped virus on the treated surface of about six hours after contact.

40. The method of claim 1 wherein the composition is applied to the nose at one or more of infratip lobule, columella, alar sidewall, facet, nostril sill, columella-labial angle, alar-facial groove, a tip defining point, facial skin surrounding the nose, and nasal cavities.

41. The method of claim 1 wherein the skin has a log reduction against a rhinovirus of at least 2.5 about five hours after contact with the composition.

42. The method of claim 1 wherein the skin has a log reduction against a rhinovirus of at least 2 about eight hours after contact with the composition.

43. The method of claim 5 wherein the skin of the mammal has a skin pH of less than 4 four hours after contact.

44. The method of claim 1 wherein the composition is applied prior to the nose, surrounding facial skin, and nasal passage being exposed to a virus.

45. The method of claim 1 wherein the composition is applied multiple times within a twenty-four hour period.

46. The method of claim 6 wherein an effective amount of the organic acid remains in the barrier layer on the nose, surrounding facial skin, and nasal passages after ten rinsings with water.

47. The method of claim 1 wherein at least 50%, by weight, of the nonvolatile components of the composition are present on the nose, surrounding facial skin, and nasal passages after three rinses with water.

48. The method of claim 1 wherein the composition further controls a fungus on the nose, surrounding facial skin, and nasal passages of the individual.

49. The method of claim 48 wherein the fungus comprises a mold, a yeast, or both.

50. The method of claim 49 wherein the fungus comprises a yeast.

51. The method of claim 50 wherein the yeast comprises Candida albicans.

52. The method of claim 48 wherein the composition imparts a log reduction of at least 4 against Candida albicans on the treated nose, surrounding facial skin, and nasal passages of the individual after a 15 second exposure to the composition.

53. A method of inhibiting transmission of a virus from a contaminated skin of a mammal to a nose or respiratory tract of the mammal by inactivating viruses, said method comprising applying to at least one of the nose, surrounding facial skin, and nasal passages of the mammal a composition comprising:
(a) about 25% to about 75%, by weight of a disinfecting alcohol;
(b) a virucidally effective amount of an organic acid; and
(c) water,
wherein the composition has a pH of about 5 or less at 25° C.

54. The method of claim 53 wherein a persistent antiviral efficacy is imparted to areas of the nose, surrounding facial skin, and nasal passages contacted by the composition.

55. The method of claim 53 wherein nonenveloped viruses are inactivated.

56. The method of claim 53 wherein rhinoviruses, picornaviruses, adenoviruses, herpes viruses, respiratory syncytial viruses, coronaviruses, influenza viruses, rotaviruses, enteroviruses, and similar pathogenic viruses are inactivated.

57. The method of claim 53 wherein acid-labile viruses are inactivated.

58. The method of claim 53 wherein rhinoviruses are inactivated.

59. A method of protecting an individual against self-infection by rhinoviruses comprising of applying a composition to the external or internal areas of the nose of the individual, or both, in an amount sufficient to eradicate rhinoviruses present on the external and/or internal areas of the nose or later introduced onto the external and/or internal areas of the nose,
said composition comprising:
(a) about 25% to about 75%, by weight of a disinfecting alcohol;
(b) a virucidally effective amount of an organic acid; and
(c) water,
wherein the composition has a pH of about 5 or less at 25° C.

60. The method of claim 59 wherein the composition is applied prior to the individual being exposed to rhinoviruses.

61. The method of claim 59 wherein the composition is applied multiple times within a twenty-four hour period.

62. A personal care kit useful for deactivating viruses which cause common colds and respiratory illnesses, said kit comprising:
(a) a composition comprising:
(i) about 25% to 75%, by weight, of a disinfecting alcohol;
(ii) about 0.05% to about 10%, by weight, of an organic acid comprising a monocarboxylic acid, a polycarboxylic acid, a polymeric acid having a plurality of carboxylic, phosphate, sulfonate, and/or sulfate moieties, anhydrides thereof, and mixtures thereof;
(iii) water, and
(iv) about 0.01% to about 5%, by weight, of a gelling agent,
wherein the composition has a pH of about 5 or less at 25° C.; and
(b) a dispensing apparatus for dispensing the composition onto one or more of a nasal area, nasal passages, and surrounding facial skin of an individual.

63. The kit of claim 62 wherein the composition is a liquid, a semisolid, a paste, a gel, or a solid.

64. The kit of claim 62 wherein the composition is dispensed as a spray, topically, drops, or an ointment.

65. The kit of claim 62 wherein the dispensing apparatus is a tissue, wipe, or fibrous or nonfibrous web material.
